SEQUENCE LISTING (108 sequences)

SEQ ID NO: 1 - protein, 5 aa, Artificial Sequence
  Synthetic heavy chain CDR1 of AbF46
DYYMS

SEQ ID NO: 2 - protein, 19 aa, Artificial Sequence
  Synthetic heavy chain CDR2 of AbF46
FIRNKANGYTTEYSASVKG

SEQ ID NO: 3 - protein, 6 aa, Artificial Sequence
  Synthetic heavy chain CDR3 of AbF46
DNWFAY

SEQ ID NO: 4 - protein, 6 aa, Artificial Sequence
  Synthetic heavy chain CDR1 of c-Met antibody
XXYYMS

SEQ ID NO: 5 - protein, 8 aa, Artificial Sequence
  Synthetic heavy chain CDR2 of c-Met antibody
RNXXNGXT

SEQ ID NO: 6 - protein, 6 aa, Artificial Sequence
  Synthetic heavy chain CDR3 of c-Met antibody
DNWLXY

SEQ ID NO: 7 - protein, 17 aa, Artificial Sequence
  Synthetic light chain CDR1 of c-Met antibody
KSSXSLLAXGNXXNYLA

SEQ ID NO: 8 - protein, 7 aa, Artificial Sequence
  Synthetic light chain CDR2 of c-Met antibody
WXSXRVX

SEQ ID NO: 9 - protein, 9 aa, Artificial Sequence
  Synthetic light chain CDR3 of c-Met antibody
XQSYSXPXT

SEQ ID NO: 10 - protein, 17 aa, Artificial Sequence
  Synthetic light chain CDR1 of AbF46
KSSQSLLASGNQNNYLA

SEQ ID NO: 11 - protein, 7 aa, Artificial Sequence
  Synthetic light chain CDR2 of AbF46
WASTRVS

SEQ ID NO: 12 - protein, 9 aa, Artificial Sequence
  Synthetic light chain CDR3 of AbF46
QQSYSAPLT

SEQ ID NO: 13 - protein, 9 aa, Artificial Sequence
  Synthetic CDR-L3 derived from L3-1 clone
QQSYSRPYT

SEQ ID NO: 14 - protein, 9 aa, Artificial Sequence
  Synthetic CDR-L3 derived from L3-2 clone
GQSYSRPLT

SEQ ID NO: 15 - protein, 9 aa, Artificial Sequence
  Synthetic CDR-L3 derived from L3-3 clone
AQSYSHPFS

SEQ ID NO: 16 - protein, 9 aa, Artificial Sequence
  Synthetic CDR-L3 derived from L3-5 clone
QQSYSRPFT

SEQ ID NO: 17 - protein, 117 aa, Artificial Sequence
  Synthetic heavy chain variable region of anti c-Met humanized antibody(huAbF46-H4)
EVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSS

SEQ ID NO: 18 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSRPYTFGQGTKVEIKR

SEQ ID NO: 19 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCGQSYSRPLTFGQGTKVEIKR

SEQ ID NO: 20 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCAQSYSHPFSFGQGTKVEIKR

SEQ ID NO: 21 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSRPFTFGQGTKVEIKR

SEQ ID NO: 22 - protein, 6 aa, Artificial Sequence
  Synthetic CDR-H1 derived from H11-4 clone
PEYYMS

SEQ ID NO: 23 - protein, 6 aa, Artificial Sequence
  Synthetic CDR-H1 derived from YC151 clone
PDYYMS

SEQ ID NO: 24 - protein, 6 aa, Artificial Sequence
  Synthetic CDR-H1 derived from YC193 clone
SDYYMS

SEQ ID NO: 25 - protein, 8 aa, Artificial Sequence
  Synthetic CDR-H2 derived from YC244 clone
RNNANGNT

SEQ ID NO: 26 - protein, 8 aa, Artificial Sequence
  Synthetic CDR-H2 derived from YC321 clone
RNKVNGYT

SEQ ID NO: 27 - protein, 6 aa, Artificial Sequence
  Synthetic CDR-H3 derived from YC354 clone
DNWLSY

SEQ ID NO: 28 - protein, 6 aa, Artificial Sequence
  Synthetic CDR-H3 derived from YC374 clone
DNWLTY

SEQ ID NO: 29 - protein, 17 aa, Artificial Sequence
  Synthetic CDR-L1 derived from L1-1 clone
KSSHSLLASGNQNNYLA

SEQ ID NO: 30 - protein, 17 aa, Artificial Sequence
  Synthetic CDR-L1 derived from L1-3 clone
KSSRSLLSSGNHKNYLA

SEQ ID NO: 31 - protein, 17 aa, Artificial Sequence
  Synthetic CDR-L1 derived from L1-4 clone
KSSKSLLASGNQNNYLA

SEQ ID NO: 32 - protein, 17 aa, Artificial Sequence
  Synthetic CDR-L1 derived from L1-12 clone
KSSRSLLASGNQNNYLA

SEQ ID NO: 33 - protein, 17 aa, Artificial Sequence
  Synthetic CDR-L1 derived from L1-22 clone
KSSHSLLASGNQNNYLA

SEQ ID NO: 34 - protein, 7 aa, Artificial Sequence
  Synthetic CDR-L2 derived from L2-9 clone
WASKRVS

SEQ ID NO: 35 - protein, 7 aa, Artificial Sequence
  Synthetic CDR-L2 derived from L2-12 clone
WGSTRVS

SEQ ID NO: 36 - protein, 7 aa, Artificial Sequence
  Synthetic CDR-L2 derived from L2-16 clone
WGSTRVP

SEQ ID NO: 37 - protein, 9 aa, Artificial Sequence
  Synthetic CDR-L3 derived from L3-32 clone
QQSYSKPFT

SEQ ID NO: 38 - DNA, 1416 bp, Artificial Sequence
  Synthetic nucleotide sequence of heavy chain of chAbF46
gaattcgccgccaccatggaatggagctgggtttttctcgtaacacttttaaatggtatccagtgtgaggtgaagctggtggagtctggaggaggcttggtacagcctgggggttctctgagactctcctgtgcaacttctgggttcaccttcactgattactacatgagctgggtccgccagcctccaggaaaggcacttgagtggttgggttttattagaaacaaagctaatggttacacaacagagtacagtgcatctgtgaagggtcggttcaccatctccagagataattcccaaagcatcctctatcttcaaatggacaccctgagagctgaggacagtgccacttattactgtgcaagagataactggtttgcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 39 - DNA, 759 bp, Artificial Sequence
  Synthetic nucleotide sequence of light chain of chAbF46
gaattcactagtgattaattcgccgccaccatggattcacaggcccaggtcctcatgttgctgctgctatcggtatctggtacctgtggagacattttgatgacccagtctccatcctccctgactgtgtcagcaggagagaaggtcactatgagctgcaagtccagtcagagtcttttagctagtggcaaccaaaataactacttggcctggcaccagcagaaaccaggacgatctcctaaaatgctgataatttgggcatccactagggtatctggagtccctgatcgcttcataggcagtggatctgggacggatttcactctgaccatcaacagtgtgcaggctgaagatctggctgtttattactgtcagcagtcctacagcgctccgctcacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 40 - protein, 447 aa, Artificial Sequence
  Synthetic amino acid sequence of H1-heavy
EVQLVESGGGLVQPGGSLRLSCAASGFTFTDYYMSWVRQAPGKGLEWLGFIRNKANGYTTEYSASVKGRFTISRDNSKNSLYLQMNSLKTEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 447 aa, Artificial Sequence
  Synthetic amino acid sequence of H3-heavy
EVQLVESGGGLVQPGGSLRLSCAASGFTFTDYYMSWVRQAPGKGLEWLGFIRNKANGYTTEYSASVKGRFTISRDNSKNSLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 447 aa, Artificial Sequence
  Synthetic amino acid sequence of H4-heavy
EVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 220 aa, Artificial Sequence
  Synthetic amino acid sequence of H1-light
DIVMTQSPDSLAVSLGERATINCKSSQSLLASGNQNNYLAWHQQKPGQPPKMLIIWASTRVSGVPDRFSGSGSGTDFTLTISSLQAEDVAVYYCQQSYSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 220 aa, Artificial Sequence
  Synthetic amino acid sequence of H2-light
DIVMTQTPLSLPVTPGEPASISCKSSQSLLASGNQNNYLAWHLQKPGQSPQMLIIWASTRVSGVPDRFSGSGSGTDFTLKISRVEAEDVGVYYCQQSYSAPLTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 220 aa, Artificial Sequence
  Synthetic amino acid sequence of H3-light
DIVMTQSPDSLAVSLGERATINCKSSQSLLASGNQNNYLAWYQQKPGQPPKLLIIWASTRVSGVPDRFSGSGSGTDFTLTISSLQAEDVAVYYCQQSYSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 219 aa, Artificial Sequence
  Synthetic amino acid sequence of H4-light
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSAPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 47 - DNA, 1350 bp, Artificial Sequence
  Synthetic nucleotide sequence of H1-heavy
gaggtgcagctggtggagtctgggggaggcttggtccagcctggagggtccctgagactctcctgtgcagcctctggattcaccttcactgactactacatgagctgggtccgccaggctccagggaaggggctggagtggttgggctttattagaaacaaagctaacggttacaccacagaatacagtgcgtctgtgaaaggcagattcaccatctcaagagataattcaaagaactcactgtatctgcaaatgaacagcctgaaaaccgaggacacggccgtgtattactgtgctagagataactggtttgcttactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 48 - DNA, 1350 bp, Artificial Sequence
  Synthetic nucleotide sequence of H3-heavy
gaggtgcagctggtggagtctgggggaggcttggtccagcctggagggtccctgagactctcctgtgcagcctctggattcaccttcactgactactacatgagctgggtccgccaggctccagggaaggggctggagtggttgggctttattagaaacaaagctaacggttacaccacagaatacagtgcgtctgtgaaaggcagattcaccatctcaagagataattcaaagaactcactgtatctgcaaatgaacagcctgcgtgctgaggacacggccgtgtattactgtgctagagataactggtttgcttactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 49 - DNA, 1350 bp, Artificial Sequence
  Synthetic nucleotide sequence of H4-heavy
gaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 50 - DNA, 669 bp, Artificial Sequence
  Synthetic nucleotide sequence of H1-light
gacatcgtgatgacccagtctccagactccctggctgtgtctctgggcgagagggccaccatcaactgcaagtccagccagagtcttttagctagcggcaaccaaaataactacttagcttggcaccagcagaaaccaggacagcctcctaagatgctcattatttgggcatctacccgggtatccggggtccctgaccgattcagtggcagcgggtctgggacagatttcactctcaccatcagcagcctgcaggctgaagatgtggcagtttattactgtcagcaatcctatagtgctcctctcacgttcggaggcggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 51 - DNA, 669 bp, Artificial Sequence
  Synthetic nucleotide sequence of H2-light
gatattgtgatgacccagactccactctccctgcccgtcacccctggagagccggcctccatctcctgcaagtccagtcagagtcttttagctagtggcaaccaaaataactacttggcctggcacctgcagaagccagggcagtctccacagatgctgatcatttgggcatccactagggtatctggagtcccagacaggttcagtggcagtgggtcaggcactgatttcacactgaaaatcagcagggtggaggctgaggatgttggagtttattactgccagcagtcctacagcgctccgctcacgttcggacagggtaccaagctggagctcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 52 - DNA, 669 bp, Artificial Sequence
  Synthetic nucleotide sequence of H3-light
gacatcgtgatgacccagtctccagactccctggctgtgtctctgggcgagagggccaccatcaactgcaagtccagccagagtcttttagctagcggcaaccaaaataactacttagcttggtaccagcagaaaccaggacagcctcctaagctgctcattatttgggcatctacccgggtatccggggtccctgaccgattcagtggcagcgggtctgggacagatttcactctcaccatcagcagcctgcaggctgaagatgtggcagtttattactgtcagcaatcctatagtgctcctctcacgttcggaggcggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 53 - DNA, 669 bp, Artificial Sequence
  Synthetic nucleotide sequence of H4-light
gatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagcgctccgctcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 54 - protein, 23 aa, Artificial Sequence
  Synthetic linker between VH and VL
GLGGLGGGGSGGGGSGGSSGVGS

SEQ ID NO: 55 - DNA, 1088 bp, Artificial Sequence
  Synthetic polynucleotide encoding scFv of huAbF46 antibody
gctagcgttttagcagaagttcaattggttgaatctggtggtggtttggttcaaccaggtggttctttgagattgtcttgtgctgcttctggttttactttcaccgattattacatgtcctgggttagacaagctccaggtaaaggtttggaatggttgggtttcattagaaacaaggctaacggttacactaccgaatattctgcttctgttaagggtagattcaccatttctagagacaactctaagaacaccttgtacttgcaaatgaactccttgagagctgaagatactgctgtttattactgcgctagagataattggtttgcttattggggtcaaggtactttggttactgtttcttctggcctcgggggcctcggaggaggaggtagtggcggaggaggctccggtggatccagcggtgtgggttccgatattcaaatgacccaatctccatcttctttgtctgcttcagttggtgatagagttaccattacttgtaagtcctcccaatctttgttggcttctggtaatcagaacaattacttggcttggcatcaacaaaaaccaggtaaagctccaaagatgttgattatttgggcttctaccagagtttctggtgttccatctagattttctggttctggttccggtactgattttactttgaccatttcatccttgcaaccagaagatttcgctacttactactgtcaacaatcttactctgctccattgacttttggtcaaggtacaaaggtcgaaatcaagagagaattcggtaagcctatccctaaccctctcctcggtctcgattctacgggtggtggtggatctggtggtggtggttctggtggtggtggttctcaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttgagtttaaac

SEQ ID NO: 56 - DNA, 5597 bp, Artificial Sequence
  Synthetic expression vector including polynucleotide encoding scFv of huAbF46 antibody
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagctaattctacttcatacattttcaattaagatgcagttacttcgctgtttttcaatattttctgttattgctagcgttttagcagaagttcaattggttgaatctggtggtggtttggttcaaccaggtggttctttgagattgtcttgtgctgcttctggttttactttcaccgattattacatgtcctgggttagacaagctccaggtaaaggtttggaatggttgggtttcattagaaacaaggctaacggttacactaccgaatattctgcttctgttaagggtagattcaccatttctagagacaactctaagaacaccttgtacttgcaaatgaactccttgagagctgaagatactgctgtttattactgcgctagagataattggtttgcttattggggtcaaggtactttggttactgtttcttctggcctcgggggcctcggaggaggaggtagtggcggaggaggctccggtggatccagcggtgtgggttccgatattcaaatgacccaatctccatcttctttgtctgcttcagttggtgatagagttaccattacttgtaagtcctcccaatctttgttggcttctggtaatcagaacaattacttggcttggcatcaacaaaaaccaggtaaagctccaaagatgttgattatttgggcttctaccagagtttctggtgttccatctagattttctggttctggttccggtactgattttactttgaccatttcatccttgcaaccagaagatttcgctacttactactgtcaacaatcttactctgctccattgacttttggtcaaggtacaaaggtcgaaatcaagagagaattcggtaagcctatccctaaccctctcctcggtctcgattctacgggtggtggtggatctggtggtggtggttctggtggtggtggttctcaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttgagtttaaacccgctgatctgataacaacagtgtagatgtaacaaaatcgactttgttcccactgtacttttagctcgtacaaaatacaatatacttttcatttctccgtaaacaacatgttttcccatgtaatatccttttctatttttcgttccgttaccaactttacacatactttatatagctattcacttctatacactaaaaaactaagacaattttaattttgctgcctgccatatttcaatttgttataaattcctataatttatcctattagtagctaaaaaaagatgaatgtgaatcgaatcctaagagaattgggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctctcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccgtgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaatttcttgaagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctggctagt

SEQ ID NO: 57 - protein, 13 aa, Artificial Sequence
  Synthetic U6-HC7 hinge
EPKSCDCHCPPCP

SEQ ID NO: 58 - DNA, 435 bp, Artificial Sequence
  Synthetic polynucleotide encoding CDR-L3 derived from L3-1 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagccgcccgtacacgttcggacagggtaccaaggtggagatcaaacgtacg

SEQ ID NO: 59 - DNA, 435 bp, Artificial Sequence
  Synthetic polynucleotide encoding CDR-L3 derived from L3-2 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtgggcagtcctacagccgtccgctcacgttcggacagggtaccaaggtggagatcaaacgtacg

SEQ ID NO: 60 - DNA, 435 bp, Artificial Sequence
  Synthetic polynucleotide encoding CDR-L3 derived from L3-3 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtgcacagtcctacagccatccgttctctttcggacagggtaccaaggtggagatcaaacgtacg

SEQ ID NO: 61 - DNA, 435 bp, Artificial Sequence
  Synthetic polynucleotide encoding CDR-L3 derived from L3-5 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagccgcccgtttacgttcggacagggtaccaaggtggagatcaaacgtacg

SEQ ID NO: 62 - protein, 462 aa, Artificial Sequence
  Synthetic polypeptide consisting of heavy chain of huAbF46-H4-A1, U6-HC7 hinge and constant region of human IgG1
MEWSWVFLVTLLNGIQCEVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - DNA, 1410 bp, Artificial Sequence
  Synthetic polynucleotide encoding polypeptide consisting of heavy chain of huAbF46-H4-A1, U6-HC7 hinge and constant region of human IgG1
gaattcgccgccaccatggaatggagctgggtttttctcgtaacacttttaaatggtatccagtgtgaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaaagctgcgattgccactgtcctccatgtccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 64 - protein, 461 aa, Artificial Sequence
  Synthetic polypeptide consisting of heavy chain of huAbF46-H4-A1, human IgG2 hinge and constant region of human IgG1
MEWSWVFLVTLLNGIQCEVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVERKCCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 1407 bp, Artificial Sequence
  Synthetic polynucleotide encoding polypeptide consisting of heavy chain of huAbF46-H4-A1, human IgG2 hinge and constant region of human IgG1
gaattcgccgccaccatggaatggagctgggtttttctcgtaacacttttaaatggtatccagtgtgaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagaggaagtgctgtgtggagtgccccccctgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 66 - protein, 460 aa, Artificial Sequence
  Synthetic polypeptide consisting of heavy chain of huAbF46-H4-A1, human IgG2 hinge and constant region of human IgG2
MEWSWVFLVTLLNGIQCEVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 67 - DNA, 1404 bp, Artificial Sequence
  Synthetic polynucleotide encoding polypeptide consisting of heavy chain of huAbF46-H4-A1, human IgG2 hinge and constant region of human IgG2
gaattcgccgccaccatggaatggagctgggtttttctcgtaacacttttaaatggtatccagtgtgaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 68 - protein, 240 aa, Artificial Sequence [sequence omitted]

SEQ ID NO: 69 - DNA, 758 bp, Artificial Sequence
  Synthetic polynucleotide encoding polypeptide consisting of light chain of huAbF46-H4-A1(H36Y) and human kappa constant region
aattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggtaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagccgcccgtacacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 70 - protein, 240 aa, Artificial Sequence
  Synthetic polypeptide consisting of light chain of huAbF46-H4-A1 and human kappa constant region
MDSQAQVLMLLLLSVSGTCGDIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSRPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 19 aa, Artificial Sequence
  Synthetic epitope in SEMA domain of c-Met
FSPQIEEPSQCPDCVVSAL

SEQ ID NO: 72 - protein, 10 aa, Artificial Sequence
  Synthetic epitope in SEMA domain of c-Met
PQIEEPSQCP

SEQ ID NO: 73 - protein, 5 aa, Artificial Sequence
  Synthetic epitope in SEMA domain of c-Met
EEPSQ

SEQ ID NO: 74 - protein, 117 aa, Artificial Sequence
  Synthetic heavy chain variable region of anti-c-Met antibody (AbF46 or huAbF46-H1)
EVQLVESGGGLVQPGGSLRLSCAASGFTFTDYYMSWVRQAPGKGLEWLGFIRNKANGYTTEYSASVKGRFTISRDNSKNSLYLQMNSLKTEDTAVYYCARDNWFAYWGQGTLVTVSS

SEQ ID NO: 75 - protein, 114 aa, Artificial Sequence
  Synthetic light chain variable region of anti-c-Met antibody (AbF46 or huAbF46-H1)
DIVMTQSPDSLAVSLGERATINCKSSQSLLASGNQNNYLAWHQQKPGQPPKMLIIWASTRVSGVPDRFSGSGSGTDFTLTISSLQAEDVAVYYCQQSYSAPLTFGGGTKVEIKR

SEQ ID NO: 76 - DNA, 1416 bp, Artificial Sequence; identical to SEQ ID NO: 38
  Synthetic nucleotide sequence of heavy chain of anti-c-Met antibody (AbF46 or huAbF46-H1)

SEQ ID NO: 77 - DNA, 759 bp, Artificial Sequence; identical to SEQ ID NO: 39
  Synthetic nucleotide sequence of light chain of anti-c-Met antibody (AbF46 or huAbF46-H1)

SEQ ID NO: 78 - DNA, 4170 bp, Artificial Sequence
  Synthetic polynucleotide encoding c-Met protein
atgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatca

SEQ ID NO: 79 - protein, 444 aa, Artificial Sequence
  Synthetic SEMA domain of c-Met
LHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNG

SEQ ID NO: 80 - protein, 451 aa, Artificial Sequence
  Synthetic PSI-IPT domain of c-Met
YTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTG

SEQ ID NO: 81 - protein, 313 aa, Artificial Sequence
  Synthetic TyrKc domain of c-Met
VHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 82 - DNA, 1332 bp, Artificial Sequence
  Synthetic polynucleotide encoding SEMA domain of c-Met
ctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggc

SEQ ID NO: 83 - DNA, 1299 bp, Artificial Sequence
  Synthetic polynucleotide encoding PSI-IPT domain of c-Met
tacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacagga

SEQ ID NO: 84 - DNA, 939 bp, Artificial Sequence
  Synthetic polynucleotide encoding TyrKc domain of c-Met
gtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatca

SEQ ID NO: 85 - protein, 13 aa, Artificial Sequence
  Synthetic heavy chain CDR3 of anti-c-Met antibody
DNWFAYWGQGTLV

SEQ ID NO: 86 - protein, 10 aa, Artificial Sequence
  Synthetic light chain CDR3 of anti-c-Met antibody
LTFGAGTKLE

SEQ ID NO: 87 - protein, 117 aa, Artificial Sequence
  Synthetic heavy chain variable region of monoclonal antibody AbF46
EVKLVESGGGLVQPGGSLRLSCATSGFTFTDYYMSWVRQPPGKALEWLGFIRNKANGYTTEYSASVKGRFTISRDNSQSILYLQMDTLRAEDSATYYCARDNWFAYWGQGTLVTVSA

SEQ ID NO: 88 - protein, 114 aa, Artificial Sequence
  Synthetic light chain variable region of anti-c-Met antibody
DILMTQSPSSLTVSAGEKVTMSCKSSQSLLASGNQNNYLAWHQQKPGRSPKMLIIWASTRVSGVPDRFIGSGSGTDFTLTINSVQAEDLAVYYCQQSYSAPLTFGAGTKLELKR

SEQ ID NO: 89 - protein, 17 aa, Artificial Sequence
  Synthetic light chain CDR3 of anti-c-Met antibody
QQSYSAPLTFGAGTKLE

SEQ ID NO: 90 - protein, 117 aa, Artificial Sequence
  Synthetic heavy chain variable region of AT-VH1
EVKLVESGGGLVQPGGSLRLSCATSGFTFTDYYMSWVRQPPGKGLEWLGFIRNKANGYTTEYSASVKGRFTISRDNSKSTLYLQMNSLRAEDSATYYCARDNWFAYWGQGTLVTVSS

SEQ ID NO: 91 - protein, 117 aa, Artificial Sequence
  Synthetic heavy chain variable region of AT-VH2
EVKLVESGGGLVQPGGSLRLSCATSGFTFTDYYMSWVRQPPGKGLEWLGFIRNKANGYTTEYSASVKGRFTISRDNSKSTLYLQMNSLRAEDTATYYCARDNWFAYWGQGTLVTVSS

SEQ ID NO: 92 - protein, 117 aa, Artificial Sequence
  Synthetic heavy chain variable region of AT-VH3
EVQLVESGGGLVQPGGSLRLSCATSGFTFTDYYMSWVRQPPGKGLEWLGFIRNKANGYTTEYSASVKGRFTISRDNSKSTLYLQMNSLRAEDTATYYCARDNWFAYWGQGTLVTVSS

SEQ ID NO: 93 - protein, 117 aa, Artificial Sequence
  Synthetic heavy chain variable region of AT-VH4
EVQLVESGGGLVQPGGSLRLSCATSGFTFTDYYMSWVRQPPGKGLEWLGFIRNKANGYTTEYSASVKGRFTISRDNSKNTLYLQMNSLRAEDTATYYCARDNWFAYWGQGTLVTVSS

SEQ ID NO: 94 - protein, 117 aa, Artificial Sequence
  Synthetic heavy chain variable region of AT-VH5
EVQLVESGGGLVQPGGSLRLSCATSGFTFTDYYMSWVRQPPGKGLEWLGFIRNKANGYTTEYSASVKGRFTISRDNSKNTLYLQMNSLRAEDTAVYYCARDNWFAYWGQGTLVTVSS

SEQ ID NO: 95 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSAPLTFGQGTKVEIKR

SEQ ID NO: 96 - protein, 113 aa, Artificial Sequence
  Synthetic light chain variable region of AT-Vk1
DILMTQSPSSLTASVGDRVTMTCKSSQSLLASGNQNNYLAWHQQKPGKAPKMLIIWASTRVSGVPDRFIGSGSGTDFTLTISSLQAEDVAVYYCQQSYSAPLTFGQGTKLEIK

SEQ ID NO: 97 - protein, 113 aa, Artificial Sequence
  Synthetic light chain variable region of AT-Vk2
DILMTQSPSSLSASVGDRVTITCKSSQSLLASGNQNNYLAWHQQKPGKAPKMLIIWASTRVSGVPDRFIGSGSGTDFTLTISSLQAEDVAVYYCQQSYSAPLTFGQGTKLEIK

SEQ ID NO: 98 - protein, 113 aa, Artificial Sequence
  Synthetic light chain variable region of AT-Vk3
DIQMTQSPSSLSASVGDRVTITCKSSQSLLASGNQNNYLAWHQQKPGKAPKMLIIWASTRVSGVPDRFIGSGSGTDFTLTISSLQAEDVAVYYCQQSYSAPLTFGQGTKLEIK

SEQ ID NO: 99 - protein, 113 aa, Artificial Sequence
  Synthetic light chain variable region of AT-Vk4
DIQMTQSPSSLSASVGDRVTITCKSSQSLLASGNQNNYLAWHQQKPGKAPKMLIIWASTRVSGVPDRFSGSGSGTDFTLTISSLQAEDVAVYYCQQSYSAPLTFGQGTKLEIK

SEQ ID NO: 100 - protein, 13 aa, Artificial Sequence
  Synthetic modified hinge region(U7-HC6)
EPSCDKHCCPPCP

SEQ ID NO: 101 - protein, 13 aa, Artificial Sequence
  Synthetic modified hinge region(U6-HC7)
EPKSCDCHCPPCP

SEQ ID NO: 102 - protein, 12 aa, Artificial Sequence
  Synthetic modified hinge region(U3-HC9)
ERKCCVECPPCP

SEQ ID NO: 103 - protein, 14 aa, Artificial Sequence
  Synthetic modified hinge region(U6-HC8)
EPRDCGCKPCPPCP

SEQ ID NO: 104 - protein, 13 aa, Artificial Sequence
  Synthetic modified hinge region(U8-HC5)
EKCDKTHTCPPCP

SEQ ID NO: 105 - protein, 15 aa, Artificial Sequence
  Synthetic human hinge region
EPKSCDKTHTCPPCP

SEQ ID NO: 106 - protein, 17 aa, Artificial Sequence
  Synthetic CDR-L1 of antibody L3-11Y
KSSQSLLAWGNQNNYLA

SEQ ID NO: 107 - protein, 114 aa, Artificial Sequence
  Synthetic amino acid sequence of light chain variable region of antibody L3-11Y
DIQMTQSPSSLSASVGDRVTITCKSSQSLLAWGNQNNYLAWYQQKPGKAPKMLIIWASTRVSGVPSRFSGSGSGTDFTLTISSLQPEDFATYYCQQSYSRPYTFGQGTKVEIKR

SEQ ID NO: 108 - protein, 220 aa, Artificial Sequence
  Synthetic amino acid sequence of light chain of antibody L3-11Y
DIQMTQSPSSLSASVGDRVTITCKSSQSLLAWGNQNNYLAWYQQKPGKAPKMLIIWASTRVSGVPSRFSGSGSGTDFTLTISSLQPEDFATYYCQQSYSRPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2015/0197542 A1
(43) Pub. Date: Jul. 16, 2015

(54) METHOD OF PURIFICATION OF ANTI-C-MET ANTIBODY

(71) Applicant: Samsung Electronics Co., Ltd., Suwon-si (KR)

(72) Inventors: Donguk Kim, Yongin-si (KR); Geun Woong Kim, Yongin-si (KR); In Hwan Lim, Suwon-si (KR)

(21) Appl. No.: 14/595,027

(22) Filed: Jan. 12, 2015

(30) Foreign Application Priority Data
Jan. 10, 2014 (KR) 10-2014-0003544

(51) Int. Cl. C07K 1/16; C12N 7/00; C07K 1/34; C07K 16/40; C07K 1/22; C07K 1/18

(52) U.S. Cl. CPC C07K 1/16; C07K 1/22; C07K 1/18; C07K 1/34; C07K 16/40; C12N 7/00; C07K 2317/565

(57) ABSTRACT

A method of purifying a protein from a protein-containing sample, a method of purifying an anti-c-Met antibody from a anti-c-Met containing sample, and an anti-c-Met antibody agent purified by such a method are provided.

### CROSS-REFERENCE TO RELATED APPLICATIONS

This application claims the benefit of Korean Patent Application No. 10-2014-0003544 filed on Jan. 10, 2014 in the Korean Intellectual Property Office, the entire disclosure of which is hereby incorporated by reference.

### INCORPORATION-BY-REFERENCE OF MATERIAL ELECTRONICALLY SUBMITTED

Incorporated by reference in its entirety herein is a computer-readable nucleotide/amino acid sequence listing submitted herewith and identified as follows: One 131,660 bytes ASCII (Text) file named “719213_ST25,” created Jan. 7, 2015.

### BACKGROUND OF THE INVENTION

1. Field

Provided is a method of purifying an anti-c-Met antibody and an anti-c-Met antibody agent purified by the method.

2. Description of the Related Art

Many useful proteins such as antibodies have been developed, and thus, economical mass purification technologies have emerged as an important issue in the field of bioengineering. In general, a recombinant plasmid including a gene encoding a protein of interest to be produced is inserted in a proper host cell (e.g., mammalian or bacterial) and cultured, to produce the protein of interest. The host cell to be used in production of the protein is a living organism, and thus, it should be cultured in a complex growth medium including various nutrients essential to cell growth, such as sugars, amino acids, growth factors, and the like.

The culture of the host cell may include a mixture of various nutrients besides the protein of interest, impurities originated from the host cell, and the like. Therefore, the development of a technique to separate the protein of interest from the host cell culture to the high yield and high purity, is required.

### BRIEF SUMMARY OF THE INVENTION

An embodiment provides a method of purifying an anti-c-Met antibody from an anti-c-Met containing sample, or a protein from a protein-containing sample, which method includes an affinity chromatography step, a cation-exchange chromatography step, and an anion-exchange chromatography step, wherein the cation-exchange chromatography is carried out under at least one of the conditions selected from the group consisting of:

a condition wherein the conductivity of an anti-c-Met antibody containing sample or a protein-containing sample loaded onto a cation-exchange chromatography material during the cation exchange chromatography step is about 5.5 mS/cm or less, for example, about 2.0 to 5.5 mS/cm, 3.0 to 5.5 mS/cm, or 4.0 to 5.5 mS/cm;

a condition wherein the conductivity of a wash buffer used in the cation-exchange chromatography step is about 7.0 mS/cm or less, for example, about 5.5 to 7.0 mS/cm, about 6.0 to 7.0 mS/cm, or about 6.5 to 7.0 mS/cm; and

a condition wherein the conductivity of a elution buffer used in the cation-exchange chromatography step is about 7.6 mS/cm or more or about 7.8 mS/cm or more, for example, about 7.6 to 9.5 mS/cm, about 7.6 to 9.0 mS/cm, about 7.6 to 8.5 mS/cm, about 7.8 to 9.5 mS/cm, 7.8 to 9.0 mS/cm, or about 7.8 to 8.5 mS/cm.

Another embodiment provides an anti-c-Met antibody agent including an anti-c-Met antibody, or protein agent including a protein, purified by a method of purifying an antibody or protein disclosed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic view illustrating a process of purification of an anti-c-Met antibody.

FIG. 2 is two graphs showing amounts of host cell protein (HCP) (upper) and yields (lower) according to the pH and salt concentration of wash buffer and elution buffer, when being eluted in the cation-exchange chromatography step.

FIG. 3 is a graph showing chromatography results from a cation-exchange chromatography step according to the salt concentration of the wash buffer.

FIGS. 4A to 4F are graphs showing SEC-HPLC chromatogram results from a cation-exchange chromatography step, under the condition of continuous pH gradient of the wash buffer (4A-4C: condition #1) or discontinuous pH gradient of the wash buffer (4D-4F: condition #2).

FIG. 5 provides two graphs showing activities of the antibody according to the level of impurities in a cation-exchange chromatography step (upper: Akt phosphorylation activity; lower: c-Met degradation activity).

FIGS. 6A to 6C are graphs showing the purity (%) of the affinity chromatography step according to the pH of the wash buffer and pH of the elution buffer (upper), amounts of HCP when being eluted (middle), and amounts of HCP when washing (lower).

FIG. 7 is a graph showing AC chromatogram results.

FIG. 8A is a graph showing SEC-HPLC chromatogram results for determining the purity in a affinity chromatography step.

FIG. 8B is a magnified view of the circled part of FIG. 8A.

FIGS. 9A and 9B are graphs illustrating SEC-HPLC chromatogram results, which show the level of formation of polymer in an anion-exchange chromatography step according to pH of the loaded sample.

FIGS. 10A to 10C are graphs illustrating SEC-HPLC chromatogram results, which show the level of formation of polymer in an anion-exchange chromatography step according to pH of the chase buffer. (10A: pH 6.5; 10B: pH 7.1; and 10C: pH 7.5).

### DETAILED DESCRIPTION OF THE INVENTION

Disclosed is a protein (in particular, an anti-c-Met antibody) purification method, wherein the method is capable of isolating an antibody at a high purity and high yield with maintaining its activities. It is suggested that, in a general purification process of an anti-c-Met antibody including three column steps (e.g., an affinity chromatography step, a cation-exchange chromatography step, and an anion-exchange chromatography step), the pH, conductivity, and/or salt concentration of a sample and/or buffers, which are loaded onto the cation exchange material and/or used in the cation-exchange chromatography step, are critical factors to affect the yield and purity of the anti-c-Met antibody, whereby optimal conditions to purify the antibody at a high purity and at a high yield are provided.

In the present description, all conductivity may be measured in any conventional manner, and they may be measured at room temperature (about 25° C.).

It is confirmed that the purification efficiency (e.g., purity, yield, etc.) of an antibody is considerably increased, when the cation-exchange chromatography step is performed under at least one condition selected from the group consisting of:

a condition that the conductivity of an anti-c-Met antibody containing sample loaded onto a cation-exchange chromatography material during the cation exchange chromatography step is about 5.5 mS/cm or less, for example, about 2.0 to about 5.5 mS/cm, about 3.0 to about 5.5 mS/cm, or about 4.0 to about 5.5 mS/cm;

a condition that the conductivity of a wash buffer used in the cation-exchange chromatography is about 7.0 mS/cm or less, for example, about 5.5 to about 7.0 mS/cm, about 6.0 to about 7.0 mS/cm, or about 6.5 to about 7.0 mS/cm; and

a condition that the conductivity of a elution buffer used in the cation-exchange chromatography is about 7.6 mS/cm or more or about 7.8 mS/cm or more, for example, about 7.6 to about 9.5 mS/cm, about 7.6 to about 9.0 mS/cm, about 7.6 to about 8.5 mS/cm, about 7.8 to about 9.5 mS/cm, about 7.8 to about 9.0 mS/cm, or about 7.8 to about 8.5 mS/cm.

The conductivity of the anti-c-Met antibody sample (e.g. an anti-c-Met antibody containing sample) loaded onto the cation-exchange chromatography material may be adjusted to the range of about 5.5 mS/cm or less, for example, about 2.0 to about 5.5 mS/cm, about 3.0 to about 5.5 mS/cm, or about 4.0 to about 5.5 mS/cm. In an embodiment, the conductivity of the anti-c-Met antibody sample loaded onto the cation-exchange chromatography material may be adjusted by a salt concentration and/or pH of the antibody sample (e.g., antibody containing sample). For example, the conductivity of the anti-c-Met antibody sample loaded onto the cation-exchange chromatography material may be adjusted to the above range by controlling the salt concentration of the antibody sample to the range of about 50 mM or less, for example, about 10 to about 50 mM, about 20 to about 50 mM, about 30 to about 50 mM, or about 40 to about 50 mM, and/or controlling the pH of the antibody sample to the range of about 5.5 or less or about 5.3 or less, for example, about 3.5 to about 5.5, about 4.5 to about 5.5, about 5.0 to about 5.5, about 3.5 to about 5.3, about 4.5 to about 5.3, or about 5.0 to about 5.3. The above range of the conductivity, salt concentration, or pH of the anti-c-Met antibody sample loaded onto the cation-exchange chromatography material may be determined considering the content of host proteins (HCP) and/or polymers formed in the antibody sample, the purity and yield of the antibody, and the connection with a virus inactivation step. For example, if the conductivity, salt concentration, or pH of the anti-c-Met antibody sample loaded onto the cation-exchange chromatography material is deviated from the range, the yield of the antibody may become decreased, and thus, the conductivity, salt concentration, or pH of the anti-c-Met antibody sample loaded to the cation-exchange chromatography material may be adjusted to the above range.

As described above, the pH of the anti-c-Met antibody sample loaded to the cation-exchange chromatography material may be about 5.5 or less or about 5.3 or less, for example, about 3.5 to about 5.5, about 4.5 to about 5.5, about 5.0 to about 5.5, about 3.5 to about 5.3, about 4.5 to about 5.3, or about 5.0 to about 5.3. The above range of the pH of the anti-c-Met antibody sample loaded to the cation-exchange chromatography material may be determined considering (i.e. taking into account) the content of HCP and/or polymers formed in the antibody sample and the purity and yield of the antibody. For example, if the pH of the anti-c-Met antibody sample loaded to the cation-exchange chromatography is lower than the range, the content of the HCP and polymers is increased, thereby decreasing the purity of the antibody; if it is higher than the range, the anti-c-Met antibody is partially released (eluted) from the resin, thereby decreasing the yield of the antibody; and thus, the range of the pH of the anti-c-Met antibody sample loaded to the cation-exchange chromatography may be adjusted to the above range.

The conductivity of the wash buffer used in the cation-exchange chromatography step may be about 7.0 mS/cm or less, for example, about 5.5 to about 7.0 mS/cm, about 6.0 to about 7.0 mS/cm, or about 6.5 to about 7.0 mS/cm. In an embodiment, the conductivity of the wash buffer used in the cation-exchange chromatograph may be adjusted to the above range by controlling the salt concentration and/or pH of the wash buffer. For example, the above range of the conductivity of the wash buffer used in the cation-exchange chromatography may be achieved by controlling the salt concentration of the wash buffer to the range of about 50 mM or less, for example, about 10 to about 50 mM, about 20 to about 50 mM, about 30 to about 50 mM, or about 40 to about 50 mM, and/or controlling the pH of the wash buffer to the range of about 5.2 to about 5.8, for example, about 5.3 to about 5.7, or about 5.4 to about 5.6. The above range of the conductivity, salt concentration, or pH of the wash buffer used in the cation-exchange chromatography may be determined considering (i.e. taking into account) the content of HCP and/or polymers formed in the antibody sample and the purity and yield of the antibody. For example, if the conductivity, salt concentration, or pH of the wash buffer used in the cation-exchange chromatography is lower than the range, the impurities such as HCP and polymers are not sufficiently removed, thereby decreasing the purity of the antibody; if it is higher than the range, the anti-c-Met antibody is partially released (eluted) from the resin, thereby decreasing the yield of the antibody; and thus, the range of the conductivity, salt concentration, or pH of the wash buffer used in the cation-exchange chromatography may be adjusted to the above range.

As described above, the range of the pH of the wash buffer used in the cation-exchange chromatography step, e.g., from about 5.2 to about 5.8, for example, from about 5.3 to about 5.7 or from about 5.4 to about 5.6, may be determined considering (i.e. taking into account) the content of HCP and/or polymers formed in the antibody sample and the purity and yield of the antibody. For example, if the pH of the wash buffer used in the cation-exchange chromatography is lower than the range, the impurities such as HCP and polymers are not sufficiently removed, thereby decreasing the purity of the antibody; if it is higher than the range, the anti-c-Met antibody is partially released (eluted) from the resin, thereby decreasing the yield of the antibody; and thus, the range of the pH of the wash buffer used in the cation-exchange chromatography may be adjusted to the above range. In one embodiment, the pH of the wash buffer may be lower than that of the pH of the anti-c-Met antibody sample loaded to the cation-exchange chromatography material.

The conductivity of the elution buffer used in the cation-exchange chromatography step may be about 7.6 mS/cm or more or about 7.8 mS/cm or more, for example, about 7.6 to about 9.5 mS/cm, about 7.6 to about 9.0 mS/cm, about 7.6 to about 8.5 mS/cm, about 7.8 to about 9.5 mS/cm, about 7.8 to about 9.0 mS/cm, or about 7.8 to about 8.5 mS/cm. In an embodiment, the conductivity of the elution buffer used in the cation-exchange chromatography may be adjusted to the above range by controlling the salt concentration and/or pH of the elution buffer. For example, the above range of the conductivity of the elution buffer used in the cation-exchange chromatography can be achieved by controlling the range of the salt concentration of the elution buffer to about 50 mM or less, for example, about 10 to about 50 mM, about 20 to about 50 mM, about 30 to about 50 mM, or about 40 to about 50 mM, and/or controlling the range of the pH of the elution buffer to about 6.6 to about 7.4, for example, about 6.8 to about 7.3 or about 7.0 to about 7.2. The above range of the conductivity, salt concentration, or pH of the elution buffer used in the cation-exchange chromatography may be determined considering (i.e. taking into account) the content of HCP and/or polymers formed in the antibody sample and the purity and yield of the antibody. For example, if the conductivity, salt concentration, or pH of the wash buffer used in the cation-exchange chromatography step is lower than the range, the anti-c-Met antibody is not sufficiently eluted from the resin, and if it is higher than the range, the impurities such as HCP are eluted together with the antibody, thereby affecting the purity of the antibody and affecting the anion-exchange chromatography process which is following the cation-exchange chromatography process. Thus, the range of the conductivity, salt concentration, or pH of the elution buffer used in the cation-exchange chromatography may be adjusted to the above range.

As described above, the range of the pH of the elution buffer used in the cation-exchange chromatography step, e.g., about 6.6 to about 7.4, for example, 6.8 to 7.3 or 7.0 to 7.2, may be determined considering the content of HCP and/or polymers formed in the antibody sample and the purity and yield of the antibody. For example, if the pH of the elution buffer used in the cation-exchange chromatography is lower than the range, the anti-c-Met antibody is not sufficiently eluted from the resin, and if it is higher than the range, the impurities such as HCP are eluted together with the antibody, thereby affecting the purity of the antibody. Thus, the range of the pH of the elution buffer used in the cation-exchange chromatography step may be adjusted to the above range.

Provided is a method of purifying an anti-c-Met antibody, wherein the method includes an affinity chromatography step, a cation-exchange chromatography step, and an anion-exchange chromatography step, wherein the cation-exchange chromatography step is performed under at least one condition selected from the group consisting of:

a condition that the conductivity of the anti-c-Met antibody sample loaded to the cation-exchange chromatography step is about 5.5 mS/cm or less, for example, about 2.0 to about 5.5 mS/cm, about 3.0 to about 5.5 mS/cm, or about 4.0 to about 5.5 mS/cm;

a condition that the conductivity of the wash buffer used in the cation-exchange chromatography step is about 7.0 mS/cm or less, for example, about 5.5 to about 7.0 mS/cm, about 6.0 to about 7.0 mS/cm, or about 6.5 to about 7.0 mS/cm; and

a condition that the conductivity of the elution buffer used in the cation-exchange chromatography step is about 7.6 mS/cm or more or about 7.8 mS/cm or more, for example, about 7.6 to about 9.5 mS/cm, about 7.6 to about 9.0 mS/cm, about 7.6 to about 8.5 mS/cm, about 7.8 to about 9.5 mS/cm, about 7.8 to about 9.0 mS/cm, or about 7.8 to about 8.5 mS/cm.

As described above, the conductivity of the antibody sample, wash buffer, and/or elution buffer may be adjusted by salt concentration and/or pH thereof. Thus, the method of purifying an anti-c-Met antibody may include an affinity chromatography step, a cation-exchange chromatography step, and an anion-exchange chromatography step, wherein the cation-exchange chromatography step may be performed under at least one condition selected from the group consisting of:

a condition that the salt concentration of the anti-c-Met antibody sample loaded to the cation-exchange chromatography material is about 50 mM or less, for example, about 10 to about 50 mM, about 20 to about 50 mM, about 30 to about 50 mM, or about 40 to about 50 mM;

a condition that the pH of the anti-c-Met antibody sample loaded onto the cation-exchange chromatography material is about 5.5 or less or about 5.3 or less, for example, about 3.5 to about 5.5, about 4.5 to about 5.5, about 5.0 to about 5.5, about 3.5 to about 5.3, about 4.5 to about 5.3, or about 5.0 to about 5.3;

a condition that the salt concentration of the wash buffer used in the cation-exchange chromatography step is about 50 mM or less, for example, about 10 to about 50 mM, about 20 to about 50 mM, about 30 to about 50 mM, or about 40 to about 50 mM;

a condition that the pH of the wash buffer used in the cation-exchange chromatography step is about 5.2 to about 5.8, for example, about 5.3 to about 5.7 or about 5.4 to about 5.6;

a condition that the salt concentration of the elution buffer used in the cation-exchange chromatography step is about 50 mM or less, for example, about 10 to about 50 mM, about 20 to about 50 mM, about 30 to about 50 mM, or about 40 to about 50 mM; and

a condition that the pH of the elution buffer used in the cation-exchange chromatography step is about 6.6 to about 7.4, for example, about 6.8 to about 7.3 or about 7.0 to about 7.2.

A particular embodiment provides a method of purifying an anti-c-Met antibody, wherein in an antibody purification method including the steps of an affinity chromatography, a cation-exchange chromatography, and an anion-exchange chromatography, the cation-exchange chromatography is performed under at least one condition selected from the group consisting of:

a condition that the conductivity of the anti-c-Met antibody sample loaded onto the cation-exchange chromatography material is about 5.5 mS/cm or less, for example, about 2.0 to about 5.5 mS/cm, about 3.0 to about 5.5 mS/cm, or about 4.0 to about 5.5 mS/cm;

a condition that the pH of the anti-c-Met antibody sample loaded to the cation-exchange chromatography is about 5.5 or less or about 5.3 or less, for example, about 3.5 to about 5.5, about 4.5 to about 5.5, about 5.0 to about 5.5, about 3.5 to about 5.3, about 4.5 to about 5.3, or about 5.0 to about 5.3;

a condition that the salt concentration of the anti-c-Met antibody sample loaded onto the cation-exchange chromatography material is about 50 mM or less, for example, about 10 to about 50 mM, about 20 to about 50 mM, about 30 to about 50 mM, or about 40 to about 50 mM;

a condition that the conductivity of the wash buffer used in the cation-exchange chromatography step is about 7.0 mS/cm or less, for example, about 5.5 to about 7.0 mS/cm, about 6.0 to about 7.0 mS/cm or about 6.5 to about 7.0 mS/cm;

a condition that the pH of the wash buffer used in the cation-exchange chromatography is about 5.2 to about 5.8, for example, about 5.3 to about 5.7 or about 5.4 to about 5.6;

a condition that the salt concentration of the wash buffer used in the cation-exchange chromatography step is about 50 mM or less, for example, about 10 to about 50 mM, about 20 to about 50 mM, about 30 to about 50 mM or about 40 to about 50 mM;

a condition that the conductivity of the elution buffer used in the cation-exchange chromatography step is about 7.6 mS/cm or more or about 7.8 mS/cm or more, for example, about 7.6 to about 9.5 mS/cm, about 7.6 to about 9.0 mS/cm, about 7.6 to about 8.5 mS/cm, about 7.8 to about 9.5 mS/cm, about 7.8 to about 9.0 mS/cm, or about 7.8 to about 8.5 mS/cm;

a condition that the pH of the elution buffer used in the cation-exchange chromatography is about 6.6 to about 7.4, for example, about 6.8 to about 7.3 or about 7.0 to about 7.2; and

a condition that the salt concentration of the elution buffer used in the cation-exchange chromatography step is about 50 mM or less, for example, about 10 to about 50 mM, about 20 to about 50 mM, about 30 to about 50 mM, or about 40 to about 50 mM.

As described above, the conductivity of the antibody sample, wash buffer, and/or elution buffer may be adjusted by salt concentration and/or pH thereof. Thus, the method of purifying an anti-c-Met antibody may include an affinity chromatography step, a cation-exchange chromatography step, and an anion-exchange chromatography step, wherein the cation-exchange chromatography step may be performed under at least one condition selected from the group consisting of:

a condition that the salt concentration of the anti-c-Met antibody sample loaded onto the cation-exchange chromatography material is about 50 mM or less, for example, about 10 to about 50 mM, about 20 to about 50 mM, about 30 to about 50 mM, or about 40 to about 50 mM;

a condition that the pH of the anti-c-Met antibody sample loaded onto the cation-exchange chromatography material is about 5.5 or less or about 5.3 or less, for example, about 3.5 to about 5.5, about 4.5 to about 5.5, about 5.0 to about 5.5, about 3.5 to about 5.3, about 4.5 to about 5.3, or about 5.0 to about 5.3;

a condition that the salt concentration of the wash buffer used in the cation-exchange chromatography step is about 50 mM or less, for example, about 10 to about 50 mM, about 20 to about 50 mM, about 30 to about 50 mM, or about 40 to about 50 mM;

a condition that the pH of the wash buffer used in the cation-exchange chromatography step is about 5.2 to about 5.8, for example, about 5.3 to about 5.7 or about 5.4 to about 5.6;

a condition that the salt concentration of the elution buffer used in the cation-exchange chromatography step is about 50 mM or less, for example, about 10 to about 50 mM, about 20 to about 50 mM, about 30 to about 50 mM, or about 40 to about 50 mM; and

a condition that the pH of the elution buffer used in the cation-exchange chromatography step is about 6.6 to about 7.4, for example, about 6.8 to about 7.3 or about 7.0 to about 7.2.

In the method of purifying an anti-c-Met antibody, except conductivity, pH, and/or salt concentration of an antibody sample to be loaded, wash buffer, and/or elution buffer, the cation-exchange chromatography step may be performed using resin (i.e., material) which is generally used in antibody purification under general conditions. For example, in the cation-exchange chromatography step, at least one resin (i.e., material) selected from the group consisting of SP Sepharose™ Fast Flow, Sepharose High Performance SP, Sepharose XL, Sepharose™ HT, SOURCE™ 15S, SOURCE™ 30S, RESOURCE™ S, Mono S™, CM Sepharose Fast Flow, Mini S, SP Sepharose Big Beads, Capto S, and the like, may be used. The cation-exchange chromatography step may essentially include a step of loading an antibody sample, a step of washing using a wash buffer, and a step of elution using an elution buffer, and besides the above steps, the cation-exchange chromatography may further include any general step. For example, the general step may be at least one elected from the group consisting of a pre-washing step, a pre-sanitization step, an equilibration step, a strip step, a post-sanitization step, a re-equilibration step, a storage step, and any combination thereof, but not be limited thereto. Among the steps, the pre-washing step and/or the pre-sanitization step may be skipped over. In a particular embodiment, the cation-exchange chromatography may include a pre-washing step, a pre-sanitization step, an equilibration step, a loading step, a washing step, an elution step, a strip step, a post-sanitization step, a re-equilibration step, and a storage step.

The pH of the anti-c-Met antibody sample loaded to the cation-exchange chromatography material may be adjusted by at least one selected from the group consisting of acetic acid, citric acid, Tris-base, HCl, NaOH, and any combination thereof. The salt concentration may be adjusted by at least one selected from the group consisting of sodium chloride (NaCl), magnesium sulfate (MgSO₄), calcium chloride (CaCl₂), ammonium sulfate, magnesium chloride (MgCl₂), potassium chloride (KCl), sodium sulfate (Na₂SO₄), and any combination thereof. The wash buffer used in the cation-exchange chromatography may include at least one selected from the group consisting of phosphate compounds (for example, sodium phosphate monobasic, sodium phosphate dibasic, etc.), acetate compounds (for example, sodium acetate, etc.), citrate compounds (for example, sodium citrate, etc.), carbonate compounds (for example, sodium carbonate, etc.), HEPES (4-(2-hydroxyethyl)-1-piperazineethanesulfonic acid), MOPS (3-(N-morpholino)propanesulfonic acid), Tris, Bis-Tris, MES (2-(N-morpholino)ethanesulfonic acid), and any combination thereof, so that adjust pH of the wash buffer to the above range. The salt concentration of the wash buffer may be adjusted by at least one selected from the group consisting of sodium chloride (NaCl), magnesium sulfate (MgSO₄), calcium chloride (CaCl₂), ammonium sulfate, magnesium chloride (MgCl₂), potassium chloride (KCl), sodium sulfate (Na₂SO₄), and any combination thereof. In a particular embodiment, the conductivity of the wash buffer may be adjusted through controlling the salt concentration of the wash buffer. For example, the wash buffer used in the cation-exchange chromatography may include sodium phosphate monobasic and sodium phosphate dibasic so that the pH of the wash buffer is within the above range, and include sodium chloride so that the salt concentration of the wash buffer is within the above range. The concentrations of sodium phosphate monobasic and sodium phosphate dibasic may be within the range of about 10 to about 50 mM and the same as or different from each other. For example, the concentrations of sodium phosphate monobasic and sodium phosphate dibasic may be the same to each other (for example, about 20 mM, respectively), but not be limited thereto.

The elution buffer used in the cation-exchange chromatography step may be at least one selected from the group consisting of phosphate compounds (for example, mono-sodium phosphate, sodium phosphate dibasic, etc.), acetate compounds (for example, sodium acetate, etc.), citrate compounds (for example, sodium citrate, etc.), carbonate compounds (for example, sodium carbonate, etc.), HEPES (4-(2-hydroxyethyl)-1-piperazineethanesulfonic acid), MOPS (3-(N-morpholino)propanesulfonic acid), Tris, Bis-Tris, MES (2-(N-morpholino)ethanesulfonic acid), and any combination thereof. The salt concentration of the elution buffer may be adjusted by at least one selected from the group consisting of sodium chloride (NaCl), magnesium sulfate (MgSO₄), calcium chloride (CaCl₂), ammonium sulfate, magnesium chloride (MgCl₂), potassium chloride (KCl), sodium sulfate (Na₂SO₄), and any combination thereof. In an embodiment, the conductivity of the elution buffer may be adjusted through controlling the salt concentration of the elution buffer. For example, the elution buffer used in the cation-exchange chromatography step may include sodium phosphate monobasic and sodium phosphate dibasic so that the pH of the wash buffer is within the above range, and include sodium chloride so that the salt concentration of the wash buffer is within the above range. The concentrations of sodium phosphate monobasic and sodium phosphate dibasic may be within the range of about 10 to about 50 mM and the same to or different from each other. For example, the concentrations of sodium phosphate monobasic and sodium phosphate dibasic may be the same to each other (for example, about 20 mM, respectively), but not be limited thereto.

In one embodiment, using the material for adjusting pH and/or salt concentration as described above, the pH and/or salt concentration of antibody sample loaded to the cation-exchange chromatography, wash buffer, and/or elution buffer can be properly adjusted, thereby adjusting the conductivity thereof to a proper range.

The method of purifying an anti-c-Met antibody may include an affinity chromatography step and an anion-exchange chromatography step, in addition to the cation-exchange chromatography step. In one embodiment, the method of purifying an anti-c-Met antibody may include an affinity chromatography step, a cation-exchange chromatography step, and an anion-exchange chromatography step in that order.

The affinity chromatography may be a protein A affinity chromatography, which is generally used in antibody purification, which may be performed using resin and conditions, which are generally employed therein. For example, the affinity chromatography may be performed using at least one resin selected from the group consisting of Protein A Sepharose, MabSelect, MabSelect Xtra, MabSelect SuRe, MabSelect SuRe LX, and any combination thereof. The affinity chromatography may essentially include a loading step of an antibody sample, a washing step using a wash buffer (at least one time, for example, one to five times or one to three times), and an elution step using an elution buffer, and besides the steps, the affinity chromatography may further include any general step. For example, the general step may be at least one selected from the group consisting of a pre-washing step, a pre-sanitization step, an equilibration step, a post-sanitization step, a re-equilibration, a storage step, and any combination thereof, but net be limited thereto.

In order to increase the purity and yield of the antibody, the elution buffer used in an affinity chromatography may be have the pH of about 3.0 to about 3.5 or about 3.1 to about 3.3. If the pH of the elution buffer is less than 3.0, polymers which are dimeric or multimeric (more than dimeric) may be generated, if the pH of the elution buffer is more than 3.5, the protein recovery rate may be decreased, and thus, the pH of the elution buffer used an affinity chromatography may be within the above range. The elution buffer may include at least one selected from the group consisting of citric acid, glycine, arginine, and any combination thereof, so that the pH of the elution buffer is within the range of about 3.0 to about 3.5 or about 3.1 to about 3.3, but not be limited thereto.

The anion-exchange chromatography may be performed using a resin (i.e., material) and conditions, which are generally used in antibody purification. For example, the anion-exchange chromatography may be performed using at least one resin selected from the group consisting of SP Sepharose™Fast Flow, Sepharose High Performance Q, Q Sepharose XL, Sepharose™ HT, SOURCE™ 15Q, SOURCE™ 30Q, RESOURCE™ Q, Mono Q™, Mini Q, Q Sepharose Big Beads, Capto adhere, and any combination thereof. The anion-exchange chromatography may include a loading step and a chase step, and besides these steps, further include any general step. For example, the general step may be at least one selected from the group consisting of a pre-sanitization step, a regeneration step, an equilibration step, a washing step (using deionized water), a post-sanitization step, a storage step, and any combination thereof, but not be limited thereto. Among these steps, the regeneration step and the deionized water washing step may be skipped over. In the chase step, a value of column volume (CV) may be from about 2 to about 4 CV, for example, about 3 CV.

As the pH of the anti-c-Met antibody sample loaded to the anion-exchange chromatography is increased, the level of formation of polymers becomes more inhibited. Therefore, considering the purity and yield of the antibody, the pH of the anti-c-Met antibody sample loaded to the anion-exchange chromatography may be adjusted to high pH range. For example, the pH of the anti-c-Met antibody sample loaded to the anion-exchange chromatography may be about 6.5 or more, about 7.1 or more, or about 7.5 or more, for example, about 6.5 to about 9, about 7 to about 8, or about 7.3 to 7.7. The anti-c-Met antibody sample loaded onto the anion-exchange chromatography material may be an eluted product (an eluate) obtained from a cation-exchange chromatography step, which is previously carried out, with no particular process or with a pH adjusting, if necessary. The pH of the anti-c-Met antibody sample loaded to the anion-exchange chromatography may be adjusted using at least one selected from the group consisting of acetic acid, citric acid, Tris-base, HCl, NaOH, and any combination thereof, but not be limited thereto.

In addition, as pH of a chase buffer used in the anion-exchange chromatography is increased, the purity of the antibody becomes increased but the content of impurities other than the antibody are also increased. Therefore, considering the above, the pH of the chase buffer may be from about 6 to about 7, for example, about 6.3 to about 6.7. The chase buffer may include at least one selected from the group consisting of phosphate compounds (for example, mono-sodium phosphate, sodium phosphate dibasic, etc.), acetate compounds (for example, sodium acetate, etc.), citrate compounds (for example, sodium citrate, etc.), carbonate compounds (for example, sodium carbonate, etc.), HEPES (4-(2-hydroxyethyl)-1-piperazineethanesulfonic acid), MOPS (3-(N-morpholino)propanesulfonic acid), Tris, Bis-Tris, MES (2-(N-morpholino)ethanesulfonic acid), and any combination thereof, so that the pH of the chase buffer is within the above range.

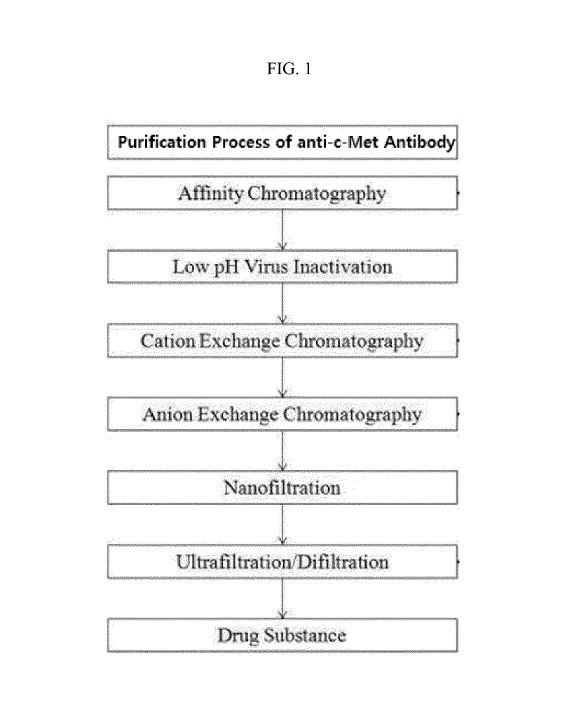

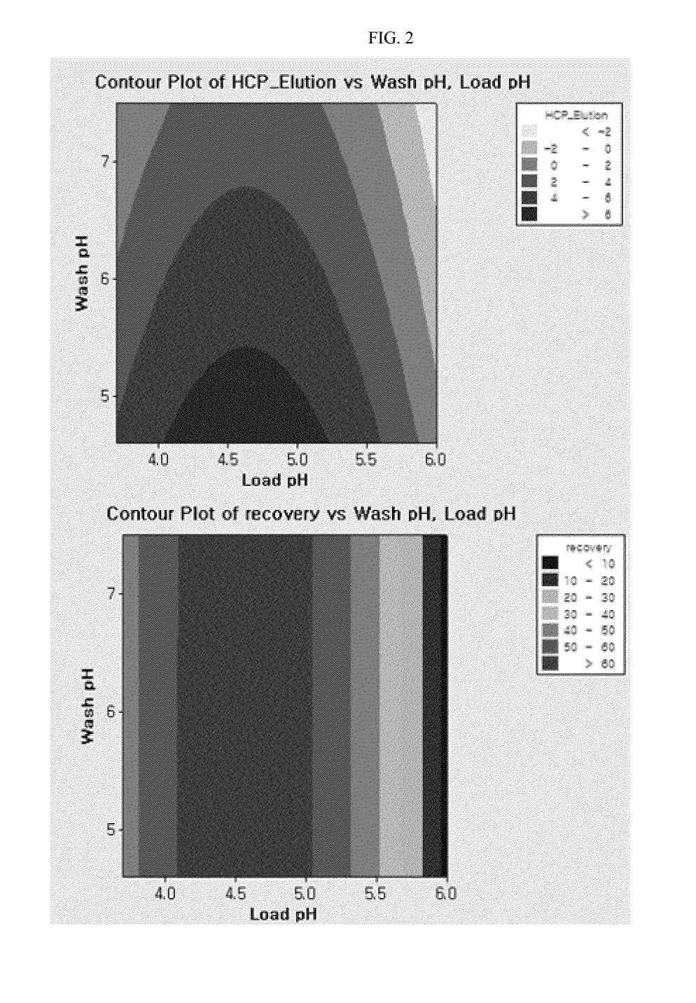

In a particular embodiment, the method of purifying an anti-c-Met antibody may further include at least one general step that is generally carried out under a general condition in an antibody purification, besides the steps of a cation-exchange chromatography, an affinity chromatography, and an anion-exchange chromatography. For example, besides the steps of a cation-exchange chromatography, an affinity chromatography, and an anion-exchange chromatography, the method of purifying an anti-c-Met antibody may further include a virus inactivation step and/or a filtration step (for example, at least one selected from the group consisting of depth filtration, microfiltration, nanofiltration, ultrafiltration, diafiltration, and any combination thereof). In a particular embodiment, the method of purifying an anti-c-Met antibody may essentially include an affinity chromatography step, a virus inactivation step, a cation-exchange chromatography step, anion-exchange chromatography, a nanofiltration step, and a ultrafiltration/diafiltration step (see FIG. 1). In particular, the method of purifying an anti-c-Met antibody may essentially include an affinity chromatography step, a virus inactivation step, a first depth filtration step, a cation-exchange chromatography step, a microfiltration step, anion-exchange chromatography, a nanofiltration step, ultrafiltration/diafiltration step, a second depth filtration step, a formulating step and a final microfiltration step, but not be limited thereto.

The anti-c-Met antibody sample used herein may refer to a sample of a host cell expressing an anti-c-Met antibody and/or a cell culture of the host cell. The anti-c-Met antibody sample loaded in each step may refer to an anti-c-Met antibody sample which passes through the previous step.

In another embodiment, provided is an anti-c-Met antibody agent prepared by the method of purifying an anti-c-Met antibody. The anti-c-Met antibody agent may possess the purity of the anti-c-Met antibody ranging 90% or more, 95% or more, 96% or more, 97% or more, 98% or more, or 99% or more. In addition, the anti-c-Met antibody agent may contain polymers (which are multimeric forms comprising at least two monomers) at the amount of 1%(w/w) or less, for example, 0.1 to 1%(w/w) or less, 0.5 to 1%(w/w), or 0.7 to 1%(w/w), and host cell proteins (HCP) at the amount of 4 ppm or less, for example, 0.5 to 4 ppm, 1 to 4 ppm, or 2 to 4 ppm on a weight basis.

The anti c-Met antibody may be any one recognizing c-Met as an antigen or any antigen-binding fragment thereof. For example, the anti-c-Met antibody may be any antibody specifically binding to c-Met thereby inducing intracellular internalization and degradation of c-Met, or any antigen-binding fragment thereof. The anti-c-Met antibody may recognize a specific region of c-Met, e.g., a specific region in the SEMA domain, as an epitope.

Unless stated otherwise, the term “anti-c-Met antibody” may be used for covering any antigen-binding region (i.e., antigen-binding fragment) as well as an anti-c-Met antibody in a complete form (e.g., a complete IgG form).

The “c-Met protein” refers to a receptor tyrosine kinase binding to hepatocyte growth factor. The c-Met proteins may be derived from any species, for example, those derived from primates such as human c-Met (e.g., GenBank Accession No. NP_{—}000236) and monkey c-Met (e.g., Macaca mulatta, GenBank Accession No. NP_{—}001162100), or those derived from rodents such as mouse c-Met (e.g., GenBank Accession No. NP_{—}032617.2) and rat c-Met (e.g., GenBank Accession No. NP_{—}113705.1). The proteins include, for example, a polypeptide encoded by the nucleotide sequence deposited under GenBank Accession No. NM_{—}000245, or a protein encoded by the polypeptide sequence deposited under GenBank Accession No. NM_{—}000236, or extracellular domains thereof. The receptor tyrosine kinase c-Met is involved in several mechanisms including cancer incidence, cancer metastasis, cancer cell migration, cancer cell penetration, angiogenesis, etc.

c-Met, a receptor for hepatocyte growth factor (HGF), may be divided into three portions: extracellular, transmembrane, and intracellular. The extracellular portion is composed of an α-subunit and a β-subunit which are linked to each other through a disulfide bond, and contains a SEMA domain responsible for binding HGF, a PSI domain (plexin-semaphorins-integrin homology domain) and an IPT domain (immunoglobulin-like fold shared by plexins and transcriptional factors domain). The SEMA domain of c-Met protein may comprise the amino acid sequence of SEQ ID NO: 79, and is an extracellular domain that functions to bind HGF. A specific region of the SEMA domain, that is, a region comprising the amino acid sequence of SEQ ID NO: 71, which corresponds to a range from amino acid residues 106 to 124 of the amino acid sequence of the SEMA domain (SEQ ID NO: 79) of c-Met protein, is a loop region between the second and the third propellers within the epitopes of the SEMA domain. The region acts as an epitope for the specific anti-c-Met antibody of the present invention.

The term “epitope” as used herein, refers to an antigenic determinant, a part of an antigen recognized by an antibody. In one embodiment, the epitope may be a region including 5 or more contiguous amino acid residues within the SEMA domain (SEQ ID NO: 79) of c-Met protein, for instance, 5 to 19 contiguous amino acid residues within the amino acid sequence of SEQ ID NO: 71. For example, the epitope may be a polypeptide including 5 to 19 contiguous amino acids selected from among partial combinations of the amino acid sequence of SEQ ID NO: 71, wherein the polypeptide comprises the amino sequence of SEQ ID NO: 73 (EEPSQ), which serves as an essential element for the epitope. For example, the epitope may be a polypeptide comprising, consisting essentially of, or consisting of the amino acid sequence of SEQ ID NO: 71, SEQ ID NO: 72, or SEQ ID NO: 73. As used herein, the phrase “contiguous amino acids” may refer to contiguous amino acid residues on the primary, secondary, or tertiary structure of a protein, wherein the contiguous amino acid residues on the secondary or tertiary structure of a protein may be consecutive or non-consecutive on the primary structure (amino acid sequence) of a protein.

The epitope comprising the amino acid sequence of SEQ ID NO: 72 corresponds to the outermost part of the loop between the second and third propellers within the SEMA domain of a c-Met protein. The epitope comprising the amino acid sequence of SEQ ID NO: 73 is a site to which the antibody or antigen-binding fragment according to one embodiment most specifically binds.

Thus, the anti-c-Met antibody may specifically bind to an epitope which includes 5 to 19 contiguous amino acids selected from among partial combinations of the amino acid sequence of SEQ ID NO: 71, including SEQ ID NO: 73 as an essential element. For example, the anti-c-Met antibody may specifically bind to an epitope comprising the amino acid sequence of SEQ ID NO: 71, SEQ ID NO: 72, or SEQ ID NO: 73.

In one embodiment, the anti-c-Met antibody or an antigen-binding fragment thereof may comprise or consist essentially of:

(i) at least one heavy chain complementarity determining region (CDR) selected from the group consisting of (a) a CDR-H1 comprising the amino acid sequence of SEQ ID NO: 4; (b) a CDR-H2 comprising the amino acid sequence of SEQ ID NO: 5, the amino acid sequence of SEQ ID NO: 2, or an amino acid sequence comprising 8-19 consecutive amino acids within the amino acid sequence of SEQ ID NO: 2 comprising amino acid residues from the 3^{rd} to 10^{th} positions of the amino acid sequence of SEQ ID NO: 2; and (c) a CDR-H3 comprising the amino acid sequence of SEQ ID NO: 6, the amino acid sequence of SEQ ID NO: 85, or an amino acid sequence comprising 6-13 consecutive amino acids within the amino acid sequence of SEQ ID NO: 85 comprising amino acid residues from the 1^{st} to 6^{th} positions of the amino acid sequence of SEQ ID NO: 85, or a heavy chain variable region comprising the at least one heavy chain complementarity determining region;

(ii) at least one light chain complementarity determining region (CDR) selected from the group consisting of (a) a CDR-L1 comprising the amino acid sequence of SEQ ID NO: 7, (b) a CDR-L2 comprising the amino acid sequence of SEQ ID NO: 8, and (c) a CDR-L3 comprising the amino acid sequence of SEQ ID NO: 9, the amino acid sequence of SEQ ID NO: 15, the amino acid sequence of SEQ ID NO: 86, or an amino acid sequence comprising 9-17 consecutive amino acids within the amino acid sequence of SEQ ID NO: 89 comprising amino acid residues from the 1^{st} to 9^{th} positions of the amino acid sequence of SEQ ID NO: 89, or a light chain variable region including the at least one light chain complementarity determining region;

(iii) a combination of the at least one heavy chain complementarity determining region and at least one light chain complementarity determining region; or

(iv) a combination of the heavy chain variable region and the light chain variable region.

Herein, the amino acid sequences of SEQ ID NOS: 4 to 9 are respectively represented by following Formulas I to VI, below:

Formula I: Xaa₁-Xaa₂-Tyr-Tyr-Met-Ser (SEQ ID NO: 4), wherein Xaa₁ is absent or Pro or Ser, and Xaa₂ is Glu or Asp,

Formula II: Arg-Asn-Xaa₃-Xaa₄-Asn-Gly-Xaa₅-Thr (SEQ ID NO: 5), wherein Xaa₃ is Asn or Lys, Xaa₄ is Ala or Val, and Xaa₅ is Asn or Thr,

Formula III: Asp-Asn-Trp-Leu-Xaa₆-Tyr (SEQ ID NO: 6), wherein Xaa₆ is Ser or Thr,

Formula IV: Lys-Ser-Ser-Xaa₇-Ser-Leu-Leu-Ala-Xaa₈-Gly-Asn-Xaa₉-Xaa₁₀-Asn-Tyr-Leu-Ala (SEQ ID NO: 7), wherein Xaa₇ is His, Arg, Gln, or Lys, Xaa₈ is Ser or Trp, Xaa₉ is His or Gln, and Xaa₁₀ is Lys or Asn,

Formula V: Trp-Xaa₁₁-Ser-Xaa₁₂-Arg-Val-Xaa₁₃ (SEQ ID NO: 8), wherein Xaa₁₁ is Ala or Gly, Xaa₁₂ is Thr or Lys, and Xaa₁₃ is Ser or Pro, and

Formula VI: Xaa₁₄-Gln-Ser-Tyr-Ser-Xaa₁₅-Pro-Xaa₁₆-Thr (SEQ ID NO: 9), wherein Xaa₁₄ is Gly, Ala, or Gln, Xaa₁₅ is Arg, His, Ser, Ala, Gly, or Lys, and Xaa₁₆ is Leu, Tyr, Phe, or Met.

In one embodiment, the CDR-H1 may include an amino acid sequence selected from the group consisting of SEQ ID NO: 1, SEQ ID NO: 22, SEQ ID NO: 23, and SEQ ID NO: 24. The CDR-H2 may include an amino acid sequence selected from the group consisting of SEQ ID NO: 2, SEQ ID NO: 25, and SEQ ID NO: 26. The CDR-H3 may include an amino acid sequence selected from the group consisting of SEQ ID NO: 3, SEQ ID NO: 27, SEQ ID NO: 28, and SEQ ID NO: 85.

The CDR-L1 may comprise an amino acid sequence selected from the group consisting of SEQ ID NO: 10, SEQ ID NO: 29, SEQ ID NO: 30, SEQ ID NO: 31, SEQ ID NO: 32, SEQ ID NO: 33, and SEQ ID NO: 106. The CDR-L2 may comprise an amino acid sequence selected from the group consisting of SEQ ID NO: 11, SEQ ID NO: 34, SEQ ID NO: 35, and SEQ ID NO: 36. The CDR-L3 may comprise an amino acid sequence selected from the group consisting of SEQ ID NO: 12, SEQ ID NO: 13, SEQ ID NO: 14, SEQ ID NO: 15, SEQ ID NO: 16, SEQ ID NO: 37, SEQ ID NO: 86, and SEQ ID NO: 89.

In another embodiment, the anti-c-Met antibody or an antigen-binding fragment thereof may comprise or consisting essentially of:

a heavy variable region comprising or consisting essentially of a polypeptide (CDR-H1) comprising an amino acid sequence selected from the group consisting of SEQ ID NO: 1, SEQ ID NO: 22, SEQ ID NO: 23, and SEQ ID NO: 24, a polypeptide (CDR-H2) comprising an amino acid sequence selected from the group consisting of SEQ ID NO: 2, SEQ ID NO: 25, and SEQ ID NO: 26, and a polypeptide (CDR-H3) comprising an amino acid sequence selected from the group consisting of SEQ ID NO: 3, SEQ ID NO: 27, SEQ ID NO: 28, and SEQ ID NO: 85; and

a light variable region comprising or consisting essentially of a polypeptide (CDR-L1) comprising an amino acid sequence selected from the group consisting of SEQ ID NO: 10, SEQ ID NO: 29, SEQ ID NO: 30, SEQ ID NO: 31, SEQ ID NO: 32, SEQ ID NO: 33, and SEQ ID NO: 106, a polypeptide (CDR-L2) comprising an amino acid sequence selected from the group consisting of SEQ ID NO: 11, SEQ ID NO: 34, SEQ ID NO: 35, and SEQ ID NO: 36, and a polypeptide (CDR-L3) comprising an amino acid sequence selected from the group consisting of SEQ ID NO: 12, SEQ ID NO: 13, SEQ ID NO: 14, SEQ ID NO: 15, SEQ ID NO: 16, SEQ ID NO: 37, SEQ ID NO: 86, and SEQ ID NO: 89.

In an embodiment, the anti-c-Met antibody or an antigen-binding fragment thereof may comprise or consist essentially of a heavy variable region comprising the amino acid sequence of SEQ ID NO: 17, SEQ ID NO: 74, SEQ ID NO: 87, SEQ ID NO: 90, SEQ ID NO: 91, SEQ ID NO: 92, SEQ ID NO: 93, or SEQ ID NO: 94, and a light variable region comprising the amino acid sequence of SEQ ID NO: 18, SEQ ID NO: 19, SEQ ID NO: 20, SEQ ID NO: 21, SEQ ID NO: 75, SEQ ID NO: 88, SEQ ID NO: 95, SEQ ID NO: 96, SEQ ID NO: 97, SEQ ID NO: 98, SEQ ID NO: 99, or SEQ ID NO: 107.

Animal-derived antibodies produced by immunizing non-immune animals with a desired antigen generally invoke immunogenicity when injected into humans for the purpose of medical treatment, and thus chimeric antibodies have been developed to inhibit such immunogenicity. Chimeric antibodies are prepared by replacing constant regions of animal-derived antibodies that cause an anti-isotype response with constant regions of human antibodies by genetic engineering. Chimeric antibodies are considerably improved in an anti-isotype response compared to animal-derived antibodies, but animal-derived amino acids still have variable regions, so that chimeric antibodies have side effects with respect to a potential anti-idiotype response. Humanized antibodies have been developed to reduce such side effects. Humanized antibodies are produced by grafting complementarity determining regions (CDR) which serve an important role in antigen binding in variable regions of chimeric antibodies into a human antibody framework.

An important aspect of CDR grafting to produce humanized antibodies is choosing the optimized human antibodies for accepting CDRs of animal-derived antibodies. Antibody databases, analysis of a crystal structure, and technology for molecule modeling are used. However, even when the CDRs of animal-derived antibodies are grafted to the most optimized human antibody framework, amino acids positioned in a framework of the animal-derived CDRs affecting antigen binding are present. Therefore, in many cases, antigen binding affinity is not maintained, and thus application of additional antibody engineering technology for recovering the antigen binding affinity is necessary.

The anti c-Met antibodies may be mouse-derived antibodies, mouse-human chimeric antibodies, humanized antibodies, or human antibodies. The antibodies or antigen-binding fragments thereof may be isolated from a living body or non-naturally occurring. The antibodies or antigen-binding fragments thereof may be recombinant or synthetic. The antibodies may be monoclonal.

An intact antibody includes two full-length light chains and two full-length heavy chains, in which each light chain is linked to a heavy chain by disulfide bonds. The antibody includes a heavy chain constant region and a light chain constant region. The heavy chain constant region is of a gamma (γ), mu (μ), alpha (α), delta (δ), or epsilon (ε) type, which may be further categorized as gamma 1 (γ1), gamma 2 (γ2), gamma 3 (γ3), gamma 4 (γ4), alpha 1 (α1), or alpha 2 (α2). The light chain constant region is of either a kappa (κ) or lambda (λ) type.

As used herein, the term “heavy chain” refers to full-length heavy chain, and fragments thereof, including a variable region V_{H} that includes amino acid sequences sufficient to provide specificity to antigens, and three constant regions, C_{H1}, C_{H2}, and C_{H3}, and a hinge. The term “light chain” refers to a full-length light chain and fragments thereof, including a variable region V_{L} that includes amino acid sequences sufficient to provide specificity to antigens, and a constant region C_{L}.

The term “complementarity determining region (CDR)” refers to an amino acid sequence found in a hyper variable region of a heavy chain or a light chain of immunoglobulin. The heavy and light chains may respectively include three CDRs (CDRH1, CDRH2, and CDRH3; and CDRL1, CDRL2, and CDRL3). The CDRs may provide contact residues that play an important role in the binding of antibodies to antigens or epitopes. The terms “specifically binding” and “specifically recognized” are well known to one of ordinary skill in the art, and indicate that an antibody and an antigen specifically interact with each other to lead to an immunological activity.

The term “antigen-binding fragment” used herein refers to fragments of an intact immunoglobulin including portions of a polypeptide including antigen-binding regions having the ability to specifically bind to the antigen. In one embodiment, the antigen-binding fragment may be selected from the group consisting of scFv, (scFv)₂, Fab, Fab′, and F(ab′)₂, but not be limited thereto.

Among the antigen-binding fragments, Fab that includes light chain and heavy chain variable regions, a light chain constant region, and a first heavy chain constant region Cₘ, includes one antigen-binding site.

The Fab′ fragment is different from the Fab fragment, in that Fab′ includes a hinge region with at least one cysteine residue at the C-terminal of C_{H1}.

The F(ab′)₂ antibody is formed through disulfide bridging of the cysteine residues in the hinge region of the Fab′ fragment. Fv is the smallest antibody fragment with only a heavy chain variable region and a light chain variable region. Recombination techniques of generating the Fv fragment are widely known in the art.

Two-chain Fv includes a heavy chain variable region and a light chain region which are linked by a non-covalent bond. Single-chain Fv generally includes a heavy chain variable region and a light chain variable region which are linked by a covalent bond via a peptide linker or linked at the C-terminals to have a dimer structure like the two-chain Fv. The peptide linker may be a polypeptide comprising 1 to 100 or 2 to 50 amino acids, wherein the amino acids may be selected from any amino acids without limitation.

The antigen-binding fragments may be attainable using protease (for example, the Fab fragment may be obtained by restricted cleavage of a whole antibody with papain, and the F(ab′)₂ fragment may be obtained by cleavage with pepsin), or may be prepared by using a genetic recombination technique.

The term “hinge region,” as used herein, refers to a region between CH1 and CH2 domains within the heavy chain of an antibody which functions to provide flexibility for the antigen-binding site.

When an animal antibody undergoes a chimerization process, the IgG1 hinge of animal origin may be replaced with a human IgG1 hinge or IgG2 hinge while the disulfide bridges between two heavy chains are reduced from three to two in number. In addition, an animal-derived IgG1 hinge is shorter than a human IgG1 hinge. Accordingly, the rigidity of the hinge is changed. Thus, a modification of the hinge region may bring about an improvement in the antigen binding efficiency of the humanized antibody. The modification of the hinge region through amino acid deletion, addition, or substitution is well-known to those skilled in the art.

In one embodiment, the anti-c-Met antibody or an antigen-binding fragment thereof may be modified by the deletion, insertion, addition, or substitution of at least one (e.g., two, three, four, five, six, seven, eight, nine, or ten) amino acid residue of the amino acid sequence of the hinge region so that it exhibits enhanced antigen-binding efficiency. For example, the antibody may include a hinge region including the amino acid sequence of SEQ ID NO: 100 (U7-HC6), 101 (U6-HC7), 102 (U3-HC9), 103 (U6-HC8), or 104 (U8-HC5), or a hinge region including the amino acid sequence of SEQ ID NO: 105 (non-modified human hinge). Preferably, the hinge region includes the amino acid sequence of SEQ ID NO: 100 or 101.

In one embodiment, the anti-c-Met antibody may be a monoclonal antibody. The monoclonal antibody may be produced by the hybridoma cell line deposited with the Korean Cell Line Research Foundation, an international depository authority located at Yungun-Dong, Jongno-Gu, Seoul, Korea, on Oct. 6, 2009, under Accession No. KCLRF-BP-00220, which binds specifically to the extracellular region of c-Met protein (refer to Korean Patent Publication No. 2011-0047698, the entire disclosure of which is incorporated herein by reference). The anti-c-Met antibody may include all the antibodies defined in Korean Patent Publication No. 2011-0047698.

In the anti-c-Met antibody, the portion of the light chain and the heavy chain portion excluding the CDRs, the light chain variable region, and the heavy chain variable region refers to the light chain constant region and the heavy chain constant region. The heavy chain constant region, the light chain constant region, and/or the region other than the CDR region, the heavy chain variable region, or the light chain variable region, may be originated from any subtype of immunoglobulin (e.g., IgA, IgD, IgE, IgG (IgG1, IgG2, IgG3, IgG4), IgM, etc.).

By way of further example, the anti-c-Met antibody may comprise or consist essentially of:

(a) a heavy chain comprising an amino acid sequence selected from the group consisting of the amino acid sequence of SEQ ID NO: 62 (wherein the amino acid sequence from amino acid residues from the 1^{st} to 17^{th} positions is a signal peptide), the amino acid sequence from the 18^{th} to 462^{nd} positions of SEQ ID NO: 62, the amino acid sequence of SEQ ID NO: 64 (wherein the amino acid sequence from the 1^{st} to 17^{th} positions is a signal peptide), the amino acid sequence from the 18^{th} to 461^{st} positions of SEQ ID NO: 64, the amino acid sequence of SEQ ID NO: 66 (wherein the amino acid sequence from the 1^{st} to 17^{th} positions is a signal peptide), and the amino acid sequence from the 18^{th} to 460^{th} positions of SEQ ID NO: 66; and

(b) a light chain comprising an amino acid sequence selected from the group consisting of the amino acid sequence of SEQ ID NO: 68 (wherein the amino acid sequence from the 1^{st} to 20^{th} positions is a signal peptide), the amino acid sequence from the 21^{st} to 240^{th} positions of SEQ ID NO: 68, the amino acid sequence of SEQ ID NO: 70 (wherein the amino acid sequence from the 1^{st} to 20^{th} positions is a signal peptide), the amino acid sequence from the 21^{st} to 240^{th} positions of SEQ ID NO: 70, and the amino acid sequence of SEQ ID NO: 108.

For example, the anti-c-Met antibody may be selected from the group consisting of:

(i) an antibody comprising (a) a heavy chain comprising the amino acid sequence of SEQ ID NO: 62 or the amino acid sequence from the 18^{th} to 462^{nd} positions of SEQ ID NO: 62 and (b) a light chain comprising the amino acid sequence of SEQ ID NO: 68 or the amino acid sequence from the 21^{st} to 240^{th} positions of SEQ ID NO: 68;

(ii) an antibody comprising (a) a heavy chain comprising the amino acid sequence of SEQ ID NO: 64 or the amino acid sequence from the 18^{th} to 461^{st} positions of SEQ ID NO: 64 and (b) a light chain including the amino acid sequence of SEQ ID NO: 68 or the amino acid sequence from the 21^{st} to 240^{th} positions of SEQ ID NO: 68;

(iii) an antibody comprising (a) a heavy chain comprising the amino acid sequence of SEQ ID NO: 66 or the amino acid sequence from the 18^{th} to 460^{th} positions of SEQ ID NO: 66 and (b) a light chain comprising the amino acid sequence of SEQ ID NO: 68 or the amino acid sequence from the 21^{st} to 240^{th} positions of SEQ ID NO: 68;

(iv) an antibody comprising (a) a heavy chain comprising the amino acid sequence of SEQ ID NO: 62 or the amino acid sequence from the 18^{th} to 462^{nd} positions of SEQ ID NO: 62 and (b) a light chain including the amino acid sequence of SEQ ID NO: 70 or the amino acid sequence from the 21^{st} to 240^{th} positions of SEQ ID NO: 70;

(v) an antibody comprising a heavy chain comprising (a) the amino acid sequence of SEQ ID NO: 64 or the amino acid sequence from the 18^{th} to 461^{st} positions of SEQ ID NO: 64 and (b) a light chain comprising the amino acid sequence of SEQ ID NO: 70 or the amino acid sequence from the 21^{st} to 240^{th} positions of SEQ ID NO: 70;

(v) an antibody comprising (a) a heavy chain comprising the amino acid sequence of SEQ ID NO: 66 or the amino acid sequence from the 18^{th} to 460^{th} positions of SEQ ID NO: 66 and (b) a light chain comprising the amino acid sequence of SEQ ID NO: 70 or the amino acid sequence from the 21^{st} to 240^{th} positions of SEQ ID NO: 70;

(vi) an antibody comprising (a) a heavy chain comprising the amino acid sequence of SEQ ID NO: 62 or the amino acid sequence from the 18^{th} to 462^{nd} positions of SEQ ID NO: 62 and (b) a light chain comprising the amino acid sequence of SEQ ID NO: 108;

(vii) an antibody comprising (a) a heavy chain comprising the amino acid sequence of SEQ ID NO: 64 or the amino acid sequence from the 18^{th} to 461^{st} positions of SEQ ID NO: 64 and (b) a light chain comprising the amino acid sequence of SEQ ID NO: 108; and

(viii) an antibody comprising (a) a heavy chain comprising the amino acid sequence of SEQ ID NO: 66 or the amino acid sequence from the 18^{th} to 460^{th} positions of SEQ ID NO: 66 and (b) a light chain comprising the amino acid sequence of SEQ ID NO: 108.

The polypeptide comprising the amino acid sequence of SEQ ID NO: 70 is a light chain including human kappa (κ) constant region, and the polypeptide comprising the amino acid sequence of SEQ ID NO: 68 is a polypeptide obtained by replacing histidine at position 62 (corresponding to position 36 of SEQ ID NO: 68 according to kabat numbering) of the polypeptide comprising the amino acid sequence of SEQ ID NO: 70 with tyrosine. The production yield of the antibodies may be increased by the replacement. The polypeptide comprising the amino acid sequence of SEQ ID NO: 108 is a polypeptide obtained by replacing serine at position 32 (position 27e according to kabat numbering in the amino acid sequence from amino acid residues 21 to 240 of SEQ ID NO: 68; positioned within CDR-L1) of SEQ ID NO: 108 with tryptophan. By such replacement, antibodies and antibody fragments including such sequences exhibit increased activities, such as c-Met biding affinity, c-Met degradation activity, Akt phosphorylation inhibition, and the like.

In another embodiment, the anti c-Met antibody may comprise a light chain complementarity determining region comprising the amino acid sequence of SEQ ID NO: 106, a light chain variable region comprising the amino acid sequence of SEQ ID NO: 107, or a light chain comprising the amino acid sequence of SEQ ID NO: 108.

In an embodiment, the anti-c-Met antibody may have an isoelectric point (pI) ranging from about 8 to about 8.5 or about 8.1 to about 8.3.

In general, characteristics of a protein purification including antibody purification may be affected by isoelectric point of the protein to be purified. Therefore, the protein capable of being purified by the anti-c-Met antibody purification method described above can be expanded to any protein (e.g., any antibody) having isoelectric point of about 8 to about 8.5 or about 8.1 to about 8.3.

Therefore, another embodiment provides a method of purifying a protein from a protein-containing sample, the method comprising performing an affinity chromatography step, a cation-exchange chromatography step, and an anion-exchange chromatography step on the protein-containing sample,

wherein the protein has an isoelectric point (pI) ranging from about 8 to about 8.5, and

the cation-exchange chromatography step is performed under at least one condition selected from the group consisting of:

(1) a condition that the protein containing sample loaded onto a cation-exchange chromatography material during the cation exchange chromatography step has a conductivity of about 5.5 mS/cm or less;

(2) a condition that the cation-exchange chromatography step uses a wash buffer with a conductivity of about 7.0 mS/cm or less; and

(3) a condition that the cation-exchange chromatography step uses an elution buffer with a conductivity of about 7.6 mS/cm or more.

All details of the anti-c-Met antibody purification method described above can be applied to the method of purifying a protein from a protein-containing sample.

The anti-c-Met antibody may be used in prevention and/or treatment of cancer. The cancer may relate to overexpression and/or abnormal activation of c-Met. The cancer may be a solid cancer or a blood cancer. For example, the cancer may be, but not limited to, one or more selected from the group consisting of squamous cell carcinoma, small-cell lung cancer, non-small-cell lung cancer, adenocarcinoma of the lung, squamous cell carcinoma of the lung, peritoneal carcinoma, skin cancer, melanoma in the skin or eyeball, rectal cancer, cancer near the anus, esophagus cancer, small intestinal tumor, endocrine gland cancer, parathyroid cancer, adrenal cancer, soft-tissue sarcoma, urethral cancer, chronic or acute leukemia, lymphocytic lymphoma, hepatoma, gastric cancer, gastrointestinal cancer, pancreatic cancer, glioblastoma, cervical cancer, ovarian cancer, liver cancer, bladder cancer, hepatocellular adenoma, breast cancer, colon cancer, large intestine cancer, endometrial carcinoma or uterine carcinoma, salivary gland tumor, kidney cancer, prostate cancer, vulvar cancer, thyroid cancer, head or neck cancer, brain cancer, osteosarcoma, and the like. The cancer may be a primary cancer or a metastatic cancer. The term “prevention and/or treatment of cancer” may be used to refer not only to inhibition of cancer cell proliferation and/or cancer cell death, but also to inhibition of metastasis and/or invasion of cancer.

The antibody purification technique provided herein is expected to produce an anti-c-Met antibody with a higher purity and yield.

### EXAMPLES

Hereafter, the present invention will be described in detail by examples.

The following examples are intended merely to illustrate the invention and are not construed to restrict the invention.

### Reference Example 1

### Construction of Anti-c-Met Antibody

1.1. Production of “AbF46”, a Mouse Antibody to c-Met

1.1.1. Immunization of Mice

To obtain immunized mice necessary for the development of a hybridoma cell line, each of five BALB/c mice (Japan SLC, Inc.), 4 to 6 weeks old, was intraperitoneally injected with a mixture of 100 μg of human c-Met/Fc fusion protein (R&D Systems) and one volume of complete Freund's adjuvant. Two weeks after the injection, a second intraperitoneal injection was conducted on the same mice with a mixture of 50 μg of human c-Met/Fc protein and one volume of incomplete Freund's adjuvant. One week after the second immunization, the immune response was finally boosted. Three days later, blood was taken from the tails of the mice and the sera were 1/1000 diluted in PBS and used to examine a titer of antibody to c-Met by ELISA. Mice found to have a sufficient antibody titer were selected for use in the cell fusion process.

1.1.2. Cell Fusion and Production of a Hybridoma

Three days before cell fusion, BALB/c mice (Japan SLC, Inc.) were immunized with an intraperitoneal injection of a mixture of 50 μg of human c-Met/Fc fusion protein and one volume of PBS. The immunized mice were anesthetized before excising the spleen from the left half of the body. The spleen was meshed to separate splenocytes which were then suspended in a culture medium (DMEM, GIBCO, Invitrogen). The cell suspension was centrifuged to recover the cell layer. The splenocytes thus obtained (1×10⁸ cells) were mixed with myeloma cells (Sp2/0) (1×10⁸ cells), followed by spinning to yield a cell pellet. The cell pellet was slowly suspended, treated with 45% polyethylene glycol (PEG) (1 mL) in DMEM for 1 min at 37° C., and supplemented with 1 mL of DMEM. To the cells was added 10 mL of DMEM over 10 min, after which incubation was conducted in a water bath at 37° C. for 5 min. Then the cell volume was adjusted to 50 mL before centrifugation. The cell pellet thus formed was resuspended at a density of 1˜2×10⁵ cells/mL in a selection medium (HAT medium). 0.1 mL of the cell suspension was allocated to each well of 96-well plates which were then incubated at 37° C. in a CO₂ incubator to establish a hybridoma cell population.

1.1.3. Selection of Hybridoma Cells Producing Monoclonal Antibodies to c-Met Protein

From the hybridoma cell population established in Reference Example 1.1.2, hybridoma cells which showed a specific response to c-Met protein were screened by ELISA using human c-Met/Fc fusion protein and human Fc protein as antigens.

Human c-Met/Fc fusion protein was seeded in an amount of 50 μL (2 μg/mL)/well to microtiter plates and allowed to adhere to the surface of each well. The antibody that remained unbound was removed by washing. For use in selecting the antibodies that do not bind c-Met but recognize Fc, human Fc protein was attached to the plate surface in the same manner.

The hybridoma cell culture obtained in Reference Example 1.1.2 was added in an amount of 50 μL to each well of the plates and incubated for 1 hour. The cells remaining unreacted were washed out with a sufficient amount of Tris-buffered saline and Tween 20 (TBST). Goat anti-mouse IgG-horseradish peroxidase (HRP) was added to the plates and incubated for 1 hour at room temperature. The plates were washed with a sufficient amount of TBST, followed by reacting the peroxidase with a substrate (OPD). Absorbance at 450 nm was measured on an ELISA reader.

Hybridoma cell lines which secrete antibodies that specifically and strongly bind to human c-Met but not human Fc were selected repeatedly. From the hybridoma cell lines obtained by repeated selection, a single clone producing a monoclonal antibody was finally separated by limiting dilution. The single clone of the hybridoma cell line producing the monoclonal antibody was deposited with the Korean Cell Line Research Foundation, an international depository authority located at Yungun-Dong, Jongno-Gu, Seoul, Korea, on Oct. 6, 2009, with Accession No. KCLRF-BP-00220 according to the Budapest Treaty (refer to Korean Patent Laid-Open Publication No. 2011-0047698).

1.1.4. Production and Purification of a Monoclonal Antibody

The hybridoma cell line obtained in Reference Example 1.1.3 was cultured in a serum-free medium, and the monoclonal antibody (AbF46) was produced and purified from the cell culture.

The hybridoma cells cultured in 50 mL of a medium (DMEM) supplemented with 10% (v/v) FBS (fetal bovine serum) were centrifuged and the cell pellet was washed twice or more with 20 mL of PBS to remove the FBS therefrom. Then, the cells were resuspended in 50 mL of DMEM and incubated for 3 days at 37° C. in a CO₂ incubator.

1.2. Construction of chAbF46, a Chimeric Antibody to c-Met

A mouse antibody is apt to elicit immunogenicity in humans. To solve this problem, chAbF46, a chimeric antibody, was constructed from the mouse antibody AbF46 produced in Reference Example 1.1.4 by replacing the constant region, but not the variable region responsible for antibody specificity, with an amino sequence of the human IgG1 antibody.

In this regard, a gene was designed to include the nucleotide sequence of “EcoRI-signal sequence-VH-NheI-CH-TGA-XhoI” (SEQ ID NO: 38) for a heavy chain and the nucleotide sequence of “EcoRI-signal sequence-VL-BsiWI-CL-TGA-XhoI” (SEQ ID NO: 39) for a light chain and synthesized. Then, a DNA fragment having the heavy chain nucleotide sequence (SEQ ID NO: 38) and a DNA fragment having the light chain nucleotide sequence (SEQ ID NO: 39) were digested with EcoRI (NEB, R0101S) and XhoI (NEB, R0146S) before cloning into a vector from the pOptiVEC™-TOPO TA Cloning Kit enclosed in an OptiCHO™ Antibody Express Kit (Cat no. 12762-019, Invitrogen), and a vector from the pcDNA™3.3-TOPO TA Cloning Kit (Cat no. 8300-01), respectively.

Each of the constructed vectors was amplified using Qiagen Maxiprep kit (Cat no. 12662), and a transient expression was performed using Freestyle™ MAX 293 Expression System (Invitrogen). 293 F cells were used for the expression and cultured in FreeStyle™ 293 Expression Medium in a suspension culture manner. At one day before the transient expression, the cells were provided in the concentration of 5×10⁵ cells/mL. After 24 hours, when the cell number reached to 1×10⁶ cells/mL, the transient expression was performed. A transfection was performed by a liposomal reagent method using Freestyle™ MAX reagent (Invitrogen), wherein in a 15 mL tube, the DNA was provided in the mixture ratio of 1:1 (heavy chain DNA:light chain DNA) and mixed with 2 mL of OptiPro™ SFM (Invitrogen) (A). In another 15 mL tube, 100 μL of Freestyle™ MAX reagent and 2 mL of OptiPro™ SFM were mixed (B), followed by mixing (A) and (B) and incubating for 15 minutes. The obtained mixture was slowly mixed with the cells provided one day before the transient expression. After completing the transfection, the cells were incubated in 130 rpm incubator for 5 days under the conditions of 37° C., 80% humidity, and 8% CO₂.

Afterwards, the cells were incubated in DMEM supplemented with 10% (v/v) FBS for 5 hours at 37° C. under a 5% CO₂ condition and then in FBS-free DMEM for 48 hours at 37° C. under a 5% CO₂ condition to produce antibody AbF46 (hereinafter referred to as “chAbF46”).

1.3. Construction of Humanized Antibody huAbF46 from Chimeric Antibody chAbF46

1.3.1. Heavy Chain Humanization

To design two domains, H1-heavy and H3-heavy, human germline genes which share the highest identity/homology with the VH gene of the mouse antibody AbF46 purified in Reference Example 1.2 were analyzed. An Ig BLAST search (www.ncbi.nlm.nih.gov/igblast/) result revealed that VH3-71 has an identity/identity/homology of 83% at the amino acid level. CDR-H1, CDR-H2, and CDR-H3 of the mouse antibody AbF46 were defined according to Kabat numbering. A design was made to introduce the CDR of the mouse antibody AbF46 into the framework of VH3-71. Hereupon, back mutations to the amino acid sequence of the mouse AbF46 were conducted at positions 30 (S→T), 48 (V→L), 73 (D→N), and 78 (T→L). Then, H1 was further mutated at positions 83 (R→K) and 84 (A→T) to finally establish H1-heavy (SEQ ID NO: 40) and H3-heavy (SEQ ID NO: 41).

For use in designing H4-heavy, human antibody frameworks were analyzed by a BLAST search. The result revealed that the VH3 subtype, known to be most stable, is very similar in framework and sequence to the mouse antibody AbF46. CDR-H1, CDR-H2, and CDR-H3 of the mouse antibody AbF46 were defined according to Kabat numbering and introduced into the VH3 subtype to construct H4-heavy (SEQ ID NO: 42).

1.3.2. Light Chain Humanization

To design two domains H1-light (SEQ ID NO: 43) and H2-light (SEQ ID NO: 44), human germline genes which share the highest identity/homology with the VH gene of the mouse antibody AbF46 were analyzed. An Ig BLAST search result revealed that VK4-1 has an identity/homology of 75% at the amino acid level. CDR-L1, CDR-L2, and CDR-L3 of the mouse antibody AbF46 were defined according to Kabat numbering. A design was made to introduce the CDR of the mouse antibody AbF46 into the framework of VK4-1. Hereupon, back mutations to the amino acid sequence of the mouse AbF46 were conducted at positions 36 (Y→H), 46 (L→M), and 49 (Y→I). Only one back mutation was conducted at position 49 (Y→I) on H2-light.

To design H3-light (SEQ ID NO: 45), human germline genes which share the highest identity/homology with the VL gene of the mouse antibody AbF46 were analyzed by a BLAST search. As a result, VK2-40 was selected. VL and VK2-40 of the mouse antibody AbF46 were found to have a identity/homology of 61% at an amino acid level. CDR-L1, CDR-L2, and CDR-L3 of the mouse antibody were defined according to Kabat numbering and introduced into the framework of VK4-1. Back mutations were conducted at positions 36 (Y→H), 46 (L→M), and 49 (Y→I) on H3-light.

For use in designing H4-light (SEQ ID NO: 46), human antibody frameworks were analyzed. A BLAST search revealed that the Vk1 subtype, known to be the most stable, is very similar in framework and sequence to the mouse antibody AbF46. CDR-L1, CDR-L2, and CDR-L3 of the mouse antibody AbF46 were defined according to Kabat numbering and introduced into the Vk1 subtype. Hereupon, back mutations were conducted at positions 36 (Y→H), 46 (L→M), and 49 (Y→I) on H4-light.

Thereafter, DNA fragments having the heavy chain nucleotide sequences (H1-heavy: SEQ ID NO: 47, H3-heavy: SEQ ID NO: 48, H4-heavy: SEQ ID NO: 49) and DNA fragments having the light chain nucleotide sequences (H1-light: SEQ ID NO: 50, H2-light: SEQ ID NO: 51, H3-light: SEQ ID NO: 52, H4-light: SEQ ID NO: 53) were digested with EcoRI (NEB, R0101S) and XhoI (NEB, R0146S) before cloning into a vector from the pOptiVEC™-TOPO TA Cloning Kit enclosed in an OptiCHO™ Antibody Express Kit (Cat no. 12762-019, Invitrogen) and a vector from the pcDNA™3.3-TOPO TA Cloning Kit (Cat no. 8300-01), respectively, so as to construct recombinant vectors for expressing a humanized antibody.

Each of the constructed vectors was amplified using Qiagen Maxiprep kit (Cat no. 12662), and a transient expression was performed using Freestyle™ MAX 293 Expression System (invitrogen). 293 F cells were used for the expression and cultured in FreeStyle™ 293 Expression Medium in a suspension culture manner. At one day before the transient expression, the cells were provided in the concentration of 5×10⁵ cells/ml, and after 24 hours, when the cell number reached to 1×10⁶ cells/mL, the transient expression was performed. A transfection was performed by a liposomal reagent method using Freestyle™ MAX reagent (Invitrogen), wherein in a 15 ml, tube, the DNA was provided in the mixture ratio of 1:1 (heavy chain DNA:light chain DNA) and mixed with 2 mL of OptiPro™ SFM (Invitrogen) (A). In another 15 mL tube, 100 μL of Freestyle™ MAX reagent and 2 mL of OptiPro™ SFM were mixed (B), followed by mixing (A) and (B) and incubating for 15 minutes. The obtained mixture was slowly mixed with the cells provided one day before the transient expression. After completing the transfection, the cells were incubated in 130 rpm incubator for 5 days under the conditions of 37 r, 80% humidity, and 8% CO₂ to produce a humanized antibody AbF46 (hereinafter, “huAbF46”). The humanized antibody huAbF46 used in the following examples comprised a combination of H4-heavy (SEQ ID NO: 42) and H4-light (SEQ ID NO: 46).

1.4. Construction of scFV Library of huAbF46 Antibody

For use in constructing an scFv of the huAbF46 antibody from the heavy and light chain variable regions of the huAbF46 antibody, a gene was designed to have the structure of “VH-linker-VL” for each of the heavy and the light chain variable region, with the linker comprising the amino acid sequence “GLGGLGGGGSGGGGSGGSSGVGS” (SEQ ID NO: 54). A polynucleotide sequence (SEQ ID NO: 55) encoding the designed scFv of huAbF46 was synthesized in Bioneer and an expression vector for the polynucleotide had the nucleotide sequence of SEQ ID NO: 56.

After expression, the product was found to exhibit specificity to c-Met.

1.5. Construction of Library Genes for Affinity Maturation

1.5.1. Selection of Target CDRs and Synthesis of Primers

The affinity maturation of huAbF46 was achieved. First, six complementary determining regions (CDRs) were defined according to Kabat numbering. The CDRs are given in Table 2 below.

TABLE 2

| CDR | Amino Acid Sequence |
|---|---|
| CDR-H1 | DYYMS (SEQ ID NO: 1) |
| CDR-H2 | FIRNKANGYTTEYSASVKG(SEQ ID NO: 2) |
| CDR-H3 | DNWFAY (SEQ ID NO: 3) |
| CDR-L1 | KSSQSLLASGNQNNYLA (SEQ ID NO: 10) |
| CDR-L2 | WASTRVS (SEQ ID NO: 11) |
| CDR-L3 | QQSYSAPLT (SEQ ID NO: 12) |

For use in the introduction of random sequences into the CDRs of the antibody, primers were designed as follows. Conventionally, N codons were utilized to introduce bases at the same ratio (25% A, 25% G, 25% C, 25% T) into desired sites of mutation. In this experiment, the introduction of random bases into the CDRs of huAbF46 was conducted in such a manner that, of the three nucleotides per codon in the wild-type polynucleotide encoding each CDR, the first and second nucleotides conserved over 85% of the entire sequence while the other three nucleotides were introduced at the same percentage (each 5%) and that the same possibility was imparted to the third nucleotide (33% G, 33% C, 33% T).

1.5.2. Construction of a Library of huAbF46 Antibodies and Affinity for c-Met

The construction of antibody gene libraries through the introduction of random sequences was carried out using the primers synthesized in the same manner as in Reference Example 1.5.1. Two PCR products were obtained using a polynucleotide covering the scFV of huAbF46 as a template, and were subjected to overlap extension PCR to give scFv library genes for huAbF46 antibodies in which only desired CDRs were mutated. Libraries targeting each of the six CDRs prepared from the scFV library genes were constructed.

The affinity for c-Met of each library was compared to that of the wildtype. Most libraries were lower in affinity for c-Met, compared to the wild-type. The affinity for c-Met was retained in some mutants.

1.6. Selection of Antibody with Improved Affinity from Libraries

After maturation of the affinity of the constructed libraries for c-Met, the nucleotide sequence of scFv from each clone was analyzed. The nucleotide sequences thus obtained are summarized in Table 3 and were converted into IgG forms. Four antibodies which were respectively produced from clones L3-1, L3-2, L3-3, and L3-5 were used in the subsequent experiments.

TABLE 3

| | Library | |
|---|---|---|
| Clone | constructed | CDR Sequence |
| H11-4 | CDR-H1 | PEYYMS (SEQ ID NO: 22) |
| YC151 | CDR-H1 | PDYYMS (SEQ ID NO: 23) |
| YC193 | CDR-H1 | SDYYMS (SEQ ID NO: 24) |
| YC244 | CDR-H2 | RNNANGNT (SEQ ID NO: 25) |
| YC321 | CDR-H2 | RNKVNGYT (SEQ ID NO: 26) |
| YC354 | CDR-H3 | DNWLSY (SEQ ID NO: 27) |
| YC374 | CDR-H3 | DNWLTY (SEQ ID NO: 28) |
| L1-1 | CDR-L1 | KSSHSLLASGNQNNYLA (SEQ ID NO: 29) |
| L1-3 | CDR-L1 | KSSRSLLSSGNHKNYLA (SEQ ID NO: 30) |
| L1-4 | CDR-L1 | KSSKSLLASGNQNNYLA (SEQ ID NO: 31) |
| L1-12 | CDR-L1 | KSSRSLLASGNQNNYLA (SEQ ID NO: 32) |
| L1-22 | CDR-L1 | KSSHSLLASGNQNNYLA (SEQ ID NO: 33) |
| L2-9 | CDR-L2 | WASKRVS (SEQ ID NO: 34) |
| L2-12 | CDR-L2 | WGSTRVS (SEQ ID NO: 35) |
| L2-16 | CDR-L2 | WGSTRVP (SEQ ID NO: 36) |
| L3-1 | CDR-L3 | QQSYSRPYT (SEQ ID NO: 13) |
| L3-2 | CDR-L3 | GQSYSRPLT (SEQ ID NO: 14) |
| L3-3 | CDR-L3 | AQSYSHPFS (SEQ ID NO: 15) |
| L3-5 | CDR-L3 | QQSYSRPFT (SEQ ID NO: 16) |
| L3-32 | CDR-L3 | QQSYSKPFT (SEQ ID NO: 37) |

1.7. Conversion of Selected Antibodies into IgG

Respective polynucleotides encoding heavy chains of the four selected antibodies were designed to have the structure of “EcoRI-signal sequence-VH-NheI-CH-XhoI” (SEQ ID NO: 38). The heavy chains of huAbF46 antibodies were used as they were because their amino acids were not changed during affinity maturation. In the case of the hinge region, however, the U6-HC7 hinge (SEQ ID NO: 57) was employed instead of the hinge of human IgG1. Genes were also designed to have the structure of “EcoRI-signal sequence-VL-BsiWI-CL-XhoI” for the light chain. Polypeptides encoding light chain variable regions of the four antibodies which were selected after the affinity maturation were synthesized in Bioneer. Then, a DNA fragment having the heavy chain nucleotide sequence (SEQ ID NO: 38) and DNA fragments having the light chain nucleotide sequences (DNA fragment comprising L3-1-derived CDR-L3: SEQ ID NO: 58, DNA fragment comprising L3-2-derived CDR-L3: SEQ ID NO: 59, DNA fragment comprising L3-3-derived CDR-L3: SEQ ID NO: 60, and DNA fragment comprising L3-5-derived CDR-L3: SEQ ID NO: 61) were digested with EcoRI (NEB, R0101S) and XhoI (NEB, R0146S) before cloning into a vector from the pOptiVEC™-TOPO TA Cloning Kit enclosed in an OptiCHO™ Antibody Express Kit (Cat no. 12762-019, Invitrogen) and a vector from the pcDNA™3.3-TOPO TA Cloning Kit (Cat no. 8300-01), respectively, so as to construct recombinant vectors for expressing affinity-matured antibodies.

Each of the constructed vectors was amplified using Qiagen Maxiprep kit (Cat no. 12662), and a transient expression was performed using Freestyle™ MAX 293 Expression System (invitrogen). 293 F cells were used for the expression and cultured in FreeStyle™ 293 Expression Medium in a suspension culture manner. At one day before the transient expression, the cells were provided in the concentration of 5×10⁵ cells/mL. After 24 hours, when the cell number reached to 1×10⁶ cells/mL, the transient expression was performed. A transfection was performed by a liposomal reagent method using Freestyle™ MAX reagent (Invitrogen), wherein in a 15 ml, tube, the DNA was provided in the mixture ratio of 1:1 (heavy chain DNA:light chain DNA) and mixed with 2 mL of OptiPro™ SFM (Invitrogen) (A). I In another 15 mL tube, 100 μL of Freestyle™ MAX reagent and 2 mL of OptiPro™ SFM were mixed (B), followed by mixing (A) and (B) and incubating for 15 minutes. The obtained mixture was slowly mixed with the cells provided one day before the transient expression. After completing the transfection, the cells were incubated in 130 rpm incubator for 5 days under the conditions of 37° C., 80% humidity, and 8% CO₂.

After centrifugation, the supernatant was applied to AKTA prime (GE Healthcare) to purify the antibody. In this regard, 100 mL of the supernatant was loaded at a flow rate of 5 mL/min to AKTA Prime equipped with a Protein A column (GE Healthcare, 17-0405-03), followed by elution with an IgG elution buffer (Thermo Scientific, 21004). The buffer was exchanged with PBS to purify four affinity-matured antibodies (hereinafter referred to as “huAbF46-H4-A1 (L3-1 origin), huAbF46-H4-A2 (L3-2 origin), huAbF46-H4-A3 (L3-3 origin), and huAbF46-H4-A5 (L3-5 origin),” respectively).

1.8. Construction of Constant Region- and/or Hinge Region-Substituted huAbF46-H4-A1

Among the four antibodies selected in Reference Example 1.7, huAbF46-H4-A1 was found to be the highest in affinity for c-Met and the lowest in Akt phosphorylation and c-Met degradation degree. In the antibody, the hinge region, or the constant region and the hinge region, were substituted.

The antibody huAbF46-H4-A1 (U6-HC7) was composed of a heavy chain comprising the heavy chain variable region of huAbF46-H4-A1, U6-HC7 hinge, and the constant region of human IgG1 constant region, and a light chain comprising the light chain variable region of huAbF46-H4-A1 and human kappa constant region. The antibody huAbF46-H4-A1 (IgG2 hinge) was composed of a heavy chain comprising a heavy chain variable region, a human IgG2 hinge region, and a human IgG1 constant region, and a light chain comprising the light chain variable region of huAbF46-H4-A1 and a human kappa constant region. The antibody huAbF46-H4-A1 (IgG2 Fc) was composed of the heavy chain variable region of huAbF46-H4-A1, a human IgG2 hinge region, and a human IgG2 constant region, and a light chain comprising the light variable region of huAbF46-H4-A1 and a human kappa constant region. Hereupon, the histidine residue at position 36 on the human kappa constant region of the light chain was changed to tyrosine in all of the three antibodies to increase antibody production.

For use in constructing the three antibodies, a polynucleotide (SEQ ID NO: 63) encoding a polypeptide (SEQ ID NO: 62) composed of the heavy chain variable region of huAbF46-H4-A1, a U6-HC7 hinge region, and a human IgG1 constant region, a polynucleotide (SEQ ID NO: 65) encoding a polypeptide (SEQ ID NO: 64) composed of the heavy chain variable region of huAbF46-H4-A1, a human IgG2 hinge region, and a human IgG1 region, a polynucleotide (SEQ ID NO: 67) encoding a polypeptide (SEQ ID NO: 66) composed of the heavy chain variable region of huAbF46-H4-A1, a human IgG2 region, and a human IgG2 constant region, and a polynucleotide (SEQ ID NO: 69) encoding a polypeptide (SEQ ID NO: 68) composed of the light chain variable region of huAbF46-H4-A1, with a tyrosine residue instead of histidine at position 36, and a human kappa constant region were synthesized in Bioneer. Then, the DNA fragments having heavy chain nucleotide sequences were inserted into a vector from the pOptiVEC™-TOPO TA Cloning Kit enclosed in an OptiCHO™ Antibody Express Kit (Cat no. 12762-019, Invitrogen) while DNA fragments having light chain nucleotide sequences were inserted into a vector from the pcDNA™3.3-TOPO TA Cloning Kit (Cat no. 8300-01) so as to construct vectors for expressing the antibodies.

Each of the constructed vectors was amplified using Qiagen Maxiprep kit (Cat no. 12662), and a transient expression was performed using Freestyle™ MAX 293 Expression System (Invitrogen). 293 F cells were used for the expression and cultured in FreeStyle™ 293 Expression Medium in a suspension culture manner. At one day before the transient expression, the cells were provided in the concentration of 5×10⁵ cells/mL. After 24 hours, when the cell number reached to 1×10⁶ cells/mL, the transient expression was performed. A transfection was performed by a liposomal reagent method using Freestyle™ MAX reagent (Invitrogen), wherein in a 15 mL tube, the DNA was provided in the mixture ratio of 1:1 (heavy chain DNA:light chain DNA) and mixed with 2 mL of OptiPro™ SFM (Invitrogen) (A). In another 15 mL tube, 100 μL of Freestyle™ MAX reagent and 2 mL of OptiPro™ SFM were mixed (B), followed by mixing (A) and (B) and incubating for 15 minutes. The obtained mixture was slowly mixed with the cells provided one day before the transient expression. After completing the transfection, the cells were incubated in 130 rpm incubator for 5 days under the conditions of 37° C., 80% humidity, and 8% CO₂.

After centrifugation, the supernatant was applied to AKTA prime (GE Healthcare) to purify the antibody. In this regard, 100 mL of the supernatant was loaded at a flow rate of 5 mL/min to AKTA Prime equipped with a Protein A column (GE Healthcare, 17-0405-03), followed by elution with IgG elution buffer (Thermo Scientific, 21004). The buffer was exchanged with PBS to finally purify three antibodies (huAbF46-H4-A1 (U6-HC7), huAbF46-H4-A1 (IgG2 hinge), and huAbF46-H4-A1 (IgG2 Fc)). Among the three antibodies, huAbF46-H4-A1 (IgG2 Fc) were representatively selected for the following examples, and referred as anti-c-Met antibody L3-1Y/IgG2. A cell culture including the anti-c-Met antibody L3-1Y/IgG2 was used in the following examples as a protein sample for antibody purification.

### Example 1

### Purification of an Anti-c-Met Antibody

A process of purification of an anti-c-Met antibody was schematically illustrated in FIG. 1.

Buffer

Buffers used in purification of an anti-c-Met antibody were summarized in Table 3:

TABLE 3

| Step | Buffer* | pH |
|---|---|---|
| AC wash I | 20 mM Sodium phosphate dibasic, | 7.5 ± 0.1 |
| | 50 mM NaCl | |
| AC wash II | 20 mM Sodium phosphate dibasic, | 7.5 ± 0.1 |
| | 1M NaCl | |
| AC Wash III | 20 mM Sodium phosphate dibasic, | 5.5 ± 0.1 |
| | 50 mM NaCl | |
| AC Elution | 20 mM Citric acid | 3.2 ± 0.1 |
| AC Sanitization | 0.5M NaOH | |
| Storage | 20% EtOH | |
| VI | 1M Citric acid | |
| Neutralization | 1M Trisma-base | |
| CIEX wash | 20 mM Sodium phosphate monobasic, | 5.5 ± 0.1 |
| | 20 mM | |
| | Sodium phosphate dibasic, 50 mM NaCl | |
| CIEX elution | 20 mM Sodium phosphate monobasic, | 7.1 ± 0.1 |
| | 20 mM | |
| | Sodium phosphate dibasic, 50 mM NaCl | |
| CIEX Strip | 1M NaCl | |
| CIEX Sanitization | 1M NaCl, 1M NaOH | |
| CIEX storage | 20% EtOH | |
| AIEX chase | 20 mM Sodium phosphate monobasic, | 6.5 ± 0.1 |
| | 20 mM | |
| | Sodium phosphate dibasic, 50 mM NaCl | |
| AIEX Strip | 0.1M Citric acid | |
| AIEX Sanitization | 1M NaOH | |
| AIEX Storage | 0.1M NaOH | |
| UF/DF | 20 mM Succinic acid, 150 mM NaCl | 6.0 ± 0.1 |
| UF/DF Sanitization | 0.5M NaOH | |
| UF/DF Storage | 0.1M NaOH | |
| Formulation | 20 mM Succinic acid, 150 mM NaCl, | 6.0 ± 0.1 |
| | 5% PolySorbate | |

All buffers used in the antibody purification were prepared before the use and stored at room temperature for a week or less. The remained buffers after using were disused. In addition, all reagents went through a microfiltration using a microfilter with 0.2 μm pore before storage.

1.1. Affinity Chromatography (AC)

18 L of the protein sample L3-1Y/IgG2, which was prepared in Reference Example 1, was subjected to an affinity chromatography using MabSelectSuRe LX resin (GE HealthCare) under the following conditions:

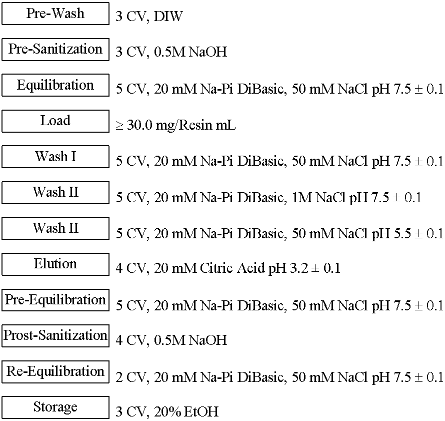

The process of selecting the particular conditions is described in Example 3 below. This step may be designed so that it can be applied to continuous column work for the production of 1000 L of clinical sample.

1.2. Low pH Virus Inactivation and Neutralization

The pH of the AC eluate obtained in Example 1.1 was titrated to the range from 3.4 to 3.6, and then, the eluate was reacted at room temperature for at least one hour or more, to perform a virus inactivation. Thereafter, the eluate was neutralized by titrating its pH to the range from 5.4 to 5.6 using 1 M Trisma-base, and reacted at 4° C. for 12 to 18.

When the eluate was reacted under low pH condition for 12 hours or more, it was found that formation of polymers is accelerated. In addition, the transparency of the protein sample (eluate) may become a little turbid depending on culture conditions (e.g., additives during culture) and/or concentration of the AC eluent, after the neutralization, which had no effect on the yield of the protein. It was confirmed that the turbid sample is due to fragments, aggregates, host cell proteins (HCP), and the like, rather than target antibodies.

1.3. Depth Filtration-1

To efficiently remove impurities induced by the neutralization, the protein sample which went through the process of Example 1.2 went through a depth filter (Sartorius Stedim biotech). The depth filter was continuously linked to a microfilter (Sartorius Stedim biotech), and the protein sample was flowed through a peristaltic pump (Sartorius Stedim biotech) wherein the flow velocity of the sample was maintained as 300 LMH, 1 L/min, or less.

The depth filter used and the conditions were summarized in Table 4:

TABLE 4

| AC column | Trial 1 | Trial 2 | units |
|---|---|---|---|
| | Depth Filtration-1 | | |
| Volume | 0.51 | 0.80 | (L) |
| Time | 1.00 | 1.92 | (hrs) |
| Flow rate | 0.51 | 0.41 | (1/hr) |
| Sartopure GF+ | Sartoscale | Cut Disc | (bar) |
| Pore rating | 1.2 | 0.65 | (μm) |
| Filter area | 0.0025 | 0.00135 | (m²) |
| Flux (average) | 300 | 300 | (lmh) |
| | Sterile Filtration | | |
| Volume | 0.507 | 1.66 | (L) |
| Pressure | 0.8 | 0.8 | (bar) |
| Sartopore2 150 | Cut Disc | Cut Disc | |
| Pore rating | 0.45 + 0.2 | 0.45 + 0.2 | (μm) |
| Filter area | 0.00135 | 0.00135 | (m²) |
| Flux (average) | 6900 | 7200 | (lmh) |

1.4. Cation Exchange Chromatography (CIEX)

A cation exchange chromatography was performed under the following conditions.

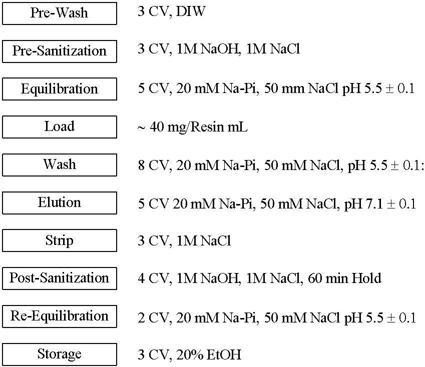

The process to select the conditions is described in Example 2 below.

1.5. Microfiltration (MF)

The CIEX eluate obtained in Example 1.4 was stored for 18 hours or less. In order to prevent propagation of microorganisms during storage and to remove possible macromolecules which may be contained in the eluate, microfilter was performed. The obtained CIEX eluate went through the microfilter (Sartorius Stedim biotech) at the flow velocity of 1 L/m using peristaltic pump. The conditions for the filtration was summarized in Table 5:

TABLE 5

| | Trial 1 | Trial 2 | |
|---|---|---|---|
| CIEX Column | MF; Sterile Filtration | | units |
| Volume | 0.844 | 2.21 | (L) |
| Pressure | 0.8 | 0.8 | (bar) |
| Sartopore2 | Cut Disc | Cut Disc | |
| Pore rating | 0.45 + 0.2 | 0.45 + 0.2 | (μm) |
| Filter area | 0.00135 | 0.00135 | (m²) |
| Flux (average) | 6200 | 6600 | (lmh) |

1.6. Anion Exchange Chromatography (AIEX)

Anion Exchange Chromatography (AIEX) was performed using Capto™ Adhere resin (GE HealthCare) under the following conditions:

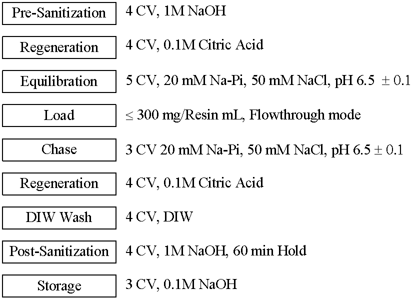

A particular process to select the conditions is described in EXAMPLE 4 below. In order to proceed with the AIEX process continuously, the equilibration step is carried out just after the post-sanitization step, so that the loading step can be followed just thereafter.

1.7. Nanofiltration (NF)

The size of the sample purified by the AIEX process was measured twice, and the size of the nanofilter (Sartorius Stedim biotech) was determined based thereon. Particular conditions of the nanofitration step are summarized in Table 6:

TABLE 6

| | Lab Scale Trials | | |
|---|---|---|---|
| | Trial 1 | Trial 2 | units |
| Category | Virus Pre-filtration | | |
| Volume | 0.072 | 0.107 | (L) |
| Pressure | 2.0 | 2.0 | (bar) |
| Sartopore2 | Minisasrt | Minisasrt | |
| Pore rating | 0.2 +0.1 | 0.2 +0.1 | (μm) |
| Filter area | 0.0005 | 0.0005 | (m²) |
| Flux (average) | 86 | 70 | (lmh) |
| | Virus filtration | | |
| Volume | 0.083 | 0.107 | (L) |
| Pressure | 2.0 | 2.0 | (bar) |
| Virosart CPV | Minisasrt | Minisasrt | |
| Pore rating | 20 nm | 20 nm | (μm) |
| Filter area | 0.0005 | 0.0005 | (m2) |
| Flux (average) | 81 | 70 | (lmh) |

1.8. Ultrafiltration/Diafiltration (UF/DF)

The sample which went through the nanofiltration process was subjected to an ultrafiltration/diafiltration (UF/DF) process. The UF/DF process was performed under the condition of TMP 0.75 bar. The conditions of the UF/DF process are summarized in Table 7:

TABLE 7

| | Lab Scale Trials | | |
|---|---|---|---|
| | Trial 1 | Trial 2 | units |
| Category | Ultrafiltration/ Diafiltration | | |
| Initial Volume | 3.5 | | (L) |
| UF Factor | 10 | | (X) |
| DF Factor | 21 | | (X) |
| Final Volume | 0.6 | | (L) |
| Sartocon | Cut Disc | | |
| Pore rating | 30 kDa | 30 kDa | (μm) |
| Filter area | 0.00135 | | (m²) |
| Flux (average) | 900 | | (lmh) |

For the UF/DF process, the steps of pre-cleaning with 0.5 M NaOH, Pre-cleaning with DIW (deionized water), pre-equilibration, sample loading, UF and DF were performed in sequence.

Particular process of each step is as follows:

Pre-Cleaning and Pre-Equilibration

The sample was subjected to a cleaning step using 0.5 M NaOH for at least 15 minutes, and DIW was flowed thereto for 30 minutes. Thereafter, the obtained sample was washed with equilibration and UF buffer until the pH and conductivity of the sample became equal to those of the buffer used.

Sample Loading

The obtained sample was loaded to membrane cassette.

Ultrafiltration (UF)

Ten (10) minutes after, an ultrafitration (UF) step was performed. It was observed that through this step, the sample can be well concentrated to generally 30 to 40 mg/ml, and maximum 60 mg/ml.

Diafiltration (DF)

A diafiltration (DF) step was sequentially performed after finishing the UF step. The DF factor was set up to about 4-folds to about 5-folds of the volume of the concentrated sample. To exactly measure the buffer change, the DF was carried out until the pH and conductivity of the sample become equal to those of the UF/DF buffer (20 mM Succinic acid, 150 mM NaCl; pH 5.9 to 6.1). The final concentration of the sample going through the UF/DF step was adjusted to 30 to 40 mg/ml.

1.9. Depth Filtration-2

To remove the impurities such as polymers from the sample going through the UF/DF process, a depth filtration-2 step was carried out before final formulation. The maximal flux of the depth filtration step was maintained as 300 LMH. The conditions of sizing by depth filter are summarized in Table 8.

TABLE 8

| | Lab Scale Trials | | |
|---|---|---|---|
| | Trial 1 | Trial 2 | units |
| Category | Depth Filtration-2 | | |
| Volume | | 0.4 | (L) |
| Time | | 1.92 | (min) |
| Flow rate | | 0.208 | (l/hr) |
| Sartopure GF+ | | Cut Disc | (bar) |
| Pore rating | | 0.65 | (μm) |
| Filter area | | 0.00135 | (m²) |
| Flux (average) | | 370 | (lmh) |

1.10. Formulation

5%(v/v) polysorbate was added to a formulation buffer at the amount of 1/100 of the final volume, so that polysorbate is present in final formulation at the concentration of 0.05%(v/v) (final formulation buffer: 20 mM Succinic acid, 150 mM NaCl, 5% PolySorbate; pH 5.9 to 6.1).

### Example 2

### Selection of Purification Conditions for Cation-Exchange Chromatography

2.1. Cation-Exchange Chromatography Process

In the purification process of an anti-c-Met antibody, second column step, cation-exchange chromatography (CIEX) step relates to removal of protein polymers, host cell proteins (HCP), and the like, and thus it is an important step to determine purity and activity of the purified antibody. The protein polymers and HCPs are important factors which can decrease bioavailability by decreasing efficacy and increasing immunogenicity, and thus they usually used as important analysis indexes in development of antibody purification process.

In the CIEX process, SP Sepharose™ Fast Flow (SPFF) resin (GE HealthCare) was employed, and the process consisted of a total of 10 steps from DIW wash step to storage step, as follows:

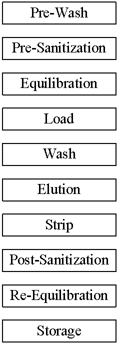

In this process, the conductivity (salt concentration) and pH of wash buffer (in wash step) was selected as main factors affecting the quality of intermediate product of this process.

2.2. Selection of Proper Conditions for Wash Buffer and Elution Buffer

The CIEX process was carried out referring to Examples 1 and 2.1, except that in the process of binding the protein (antibody) to a resin (i.e., material), washing the resin, and then recovering the protein, the example was designed so that the elution was induced not by salt but by pH. In the pH elution, the following two conditions are required: first, when the target protein is loaded, the protein should completely bind to the resin without passing through, to minimalize the loss of the protein, and second, the protein sample should be washed at a proper pH, to effectively remove the impurities. An optimal pH satisfying the two conditions at the same time was employed as a pH of the wash buffer, and the pH which is slightly higher than the pH of wash buffer was employed as a pH of the elution buffer.

The experiment conditions are summarized in Table 9:

TABLE 9

| LFR (linear | VFR (volumetric | | |
|---|---|---|---|
| flow rate; cm/h) | flow rate; ml/min) | Temp.(° C.) | Column |
| 762 | 10 | 20 to 22 | Tricorn 10/100 & 10 cm bed height |

The pH and salt concentration (conductivity) of the wash buffer were selected so as to maximize its washing ability in the cation-exchange chromatography step. For this, sodium phosphate monobasic and sodium phosphate dibasic were mixed to produce sodium phosphate buffers having continuous pH gradation. To observe the effect of salt concentration, 0 mM, 50 mM, or 100 mM NaCl was added to each sodium phosphate buffer. Gradient length was set up to basically 120 CV. The conditions of the three experiments are summarized in Table 10:

TABLE 10

| Condition | | | Salt |
|---|---|---|---|
| No. | Buffer A | Buffer B | (NaCl) |
| 1 | 20 mM Na-Pi | 20 mM Na-Pi | 0 mM |
| | monobasic (pH 4.5) | Dibasic (pH 9.4) | |
| 2 | 20 mM Na-Pi | 20 mM Na-Pi | 50 mM |
| | monobasic (pH 4.5) | Dibasic (pH 9.4) | |
| 3 | 20 mM Na-Pi | 20 mM Na-Pi | 100 mM |
| | monobasic (pH 4.5) | Dibasic (pH 9.4) | |

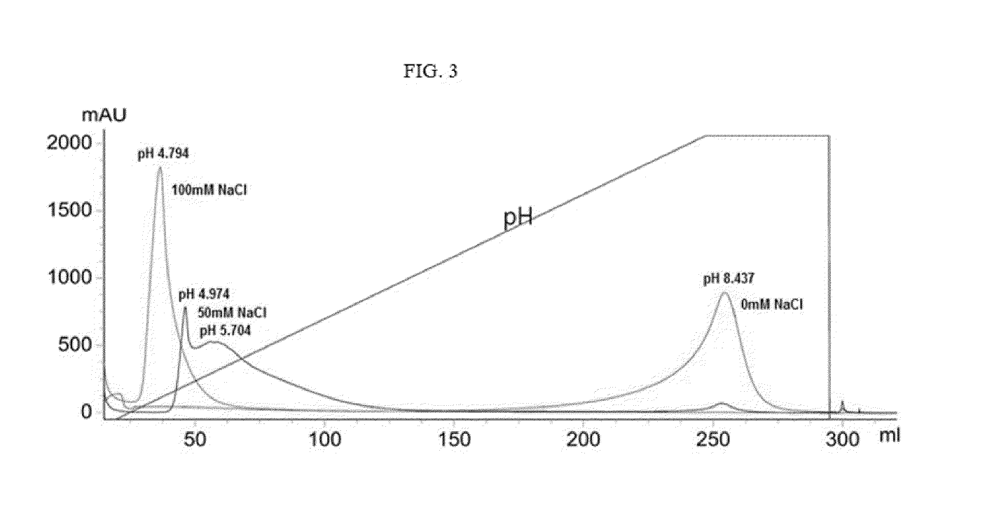

The protein was washed and eluted under the three conditions and chromatogram results depending on salt concentration and pH were obtained. The obtained chromatogram results were illustrated in FIG. 3. In FIG. 3, the linear graph going crossing from left-lower end to right-upper end exhibits the amount of eluted protein depending on the pH (left-lower end: pH 4.5; right-upper end: pH 9.4). As shown in FIG. 3, it was observed that when the salt concentrate of the wash buffer is 50 mM or 100 mM, the protein was eluted under a specific pH condition. In particular, when the salt concentration is 50 mM, the protein peak was divided to two peaks, wherein one of the two peaks is impurity peak and the other is antibody protein peak, indicating that it is easy to recover pure antibody protein from the separate antibody protein peak. On the other hand, when the salt concentration is 100 mM, the antibody did not bind to the resin and was immediately eluted together with impurities, and when the salt concentration is 0 mM, both of the antibody and purities are not eluted and remained at the resin, where in the two cases, it is not possible to obtain pure antibody. Therefore, the proper salt concentration of the wash buffer can be determined as about 50 mM. According to the changes in the graph of FIG. 3, when the salt concentration of the wash buffer is higher than 50 mM, the loss of the antibody protein becomes increased, and thus, it may be preferable that the salt concentration of the wash buffer is adjusted to 50 mM or less. In addition, when the salt concentration of the wash buffer is 50 mM, the pH corresponding to the antibody protein peak was about 5.7.

In the above experiment, when the salt concentration is 50 mM, the conditions of the wash buffer and elution buffer are summarized in Table 11:

TABLE 11

| | pH | conductivity (mS/cm) |
|---|---|---|
| wash buffer | 5.5 | 6.9 |
| elution buffer | 7.1 | 7.8 |

2.3. Experiment for the Ability to Remove Polymers Depending on pH Change

On the basis of the results of the above experiments, the pH of the loading protein sample and the wash buffer was determined from about 5.0 to about 6.5, and the salt concentration of the wash buffer was adjusted to about 50 mM. In some case, the polymers are observed in the eluted sample, which affects not only the purity but also the activity of the antibody, and thus, the proper pH was determined by more minutely analyzing the results of the preceding experiments.

As shown in following Table 12, 20 mM Na-Pi monobasic and 20 mM Na-Pi monobasic dibasic were mixed to establish the conditions of continuous pH gradation (Condition #1) and discontinuous pH gradation (Condition #2) for elution experiment.

TABLE 12

| | Buffer A | Buffer B | Salt |
|---|---|---|---|
| Condition #1 | 20 mM Na-Pi | 20 mM Na-Pi | 50 mM |
| (Gradient elution) | Monobasic | Dibasic | |
| Condition #2 | 20 mM Na-Pi | 20 mM Na-Pi | 50 mM |
| (Step elution) | Monobasic | Dibasic | |

Firstly, according to Condition#1, the pH at which the anti-c-Met antibody was not eluted was determined using the continuous pH gradient. As determined in the preceding experiments, the pH range at which the anti-c-Met antibody was not eluted was determined as less than 6.5, and it was observed that when the pH is higher than 6.5 or more, the target protein (antibody) is eluted from the resin. Thereafter, according to Condition #2 (reducing the range), an experiment using discontinuous pH gradient was performed. The range of pH gradient was from pH 4 to pH 8.

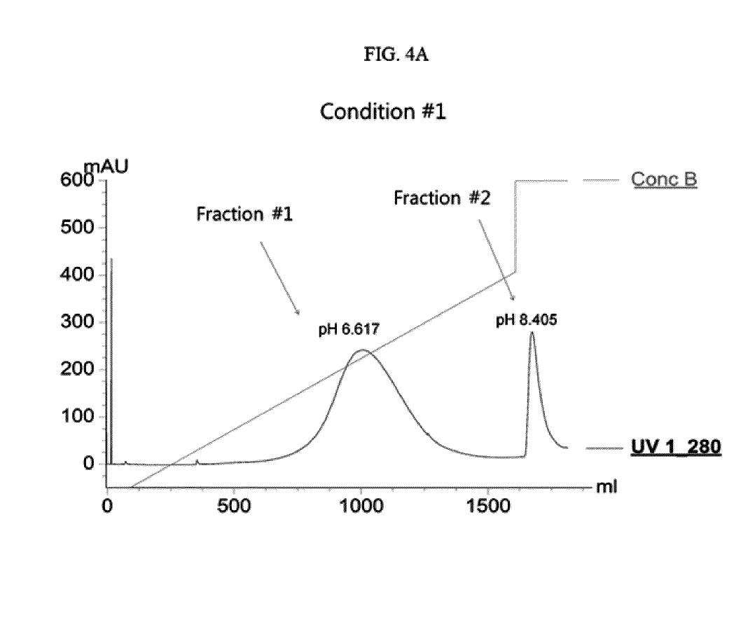

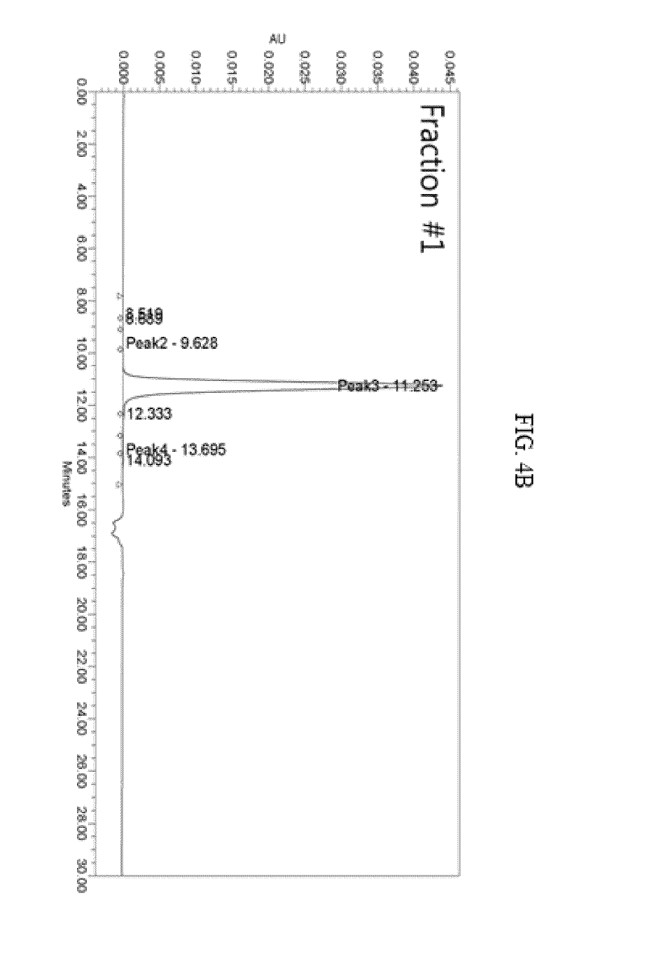

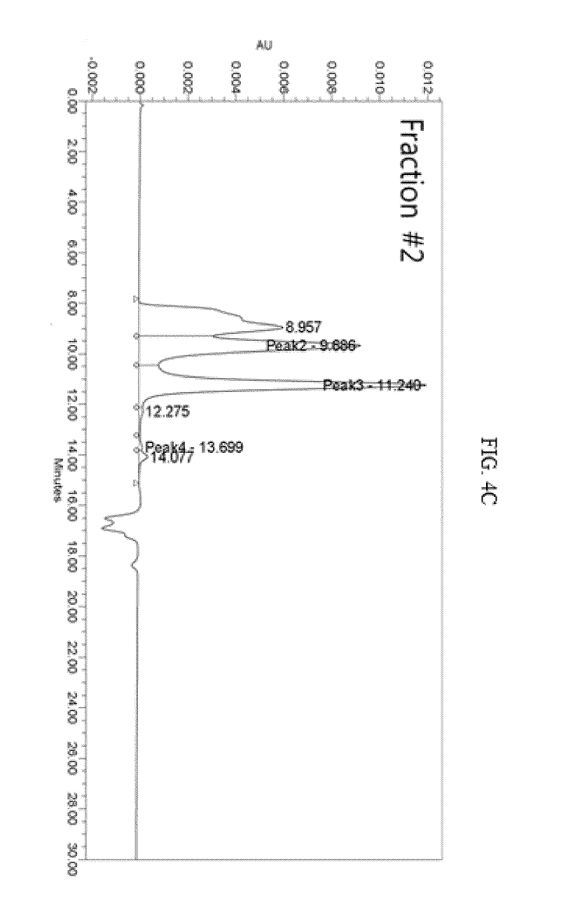

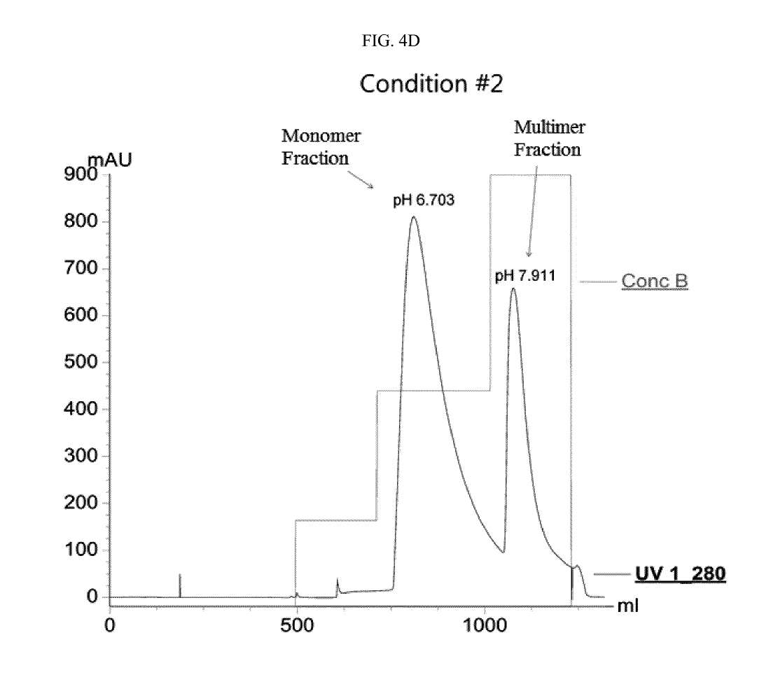

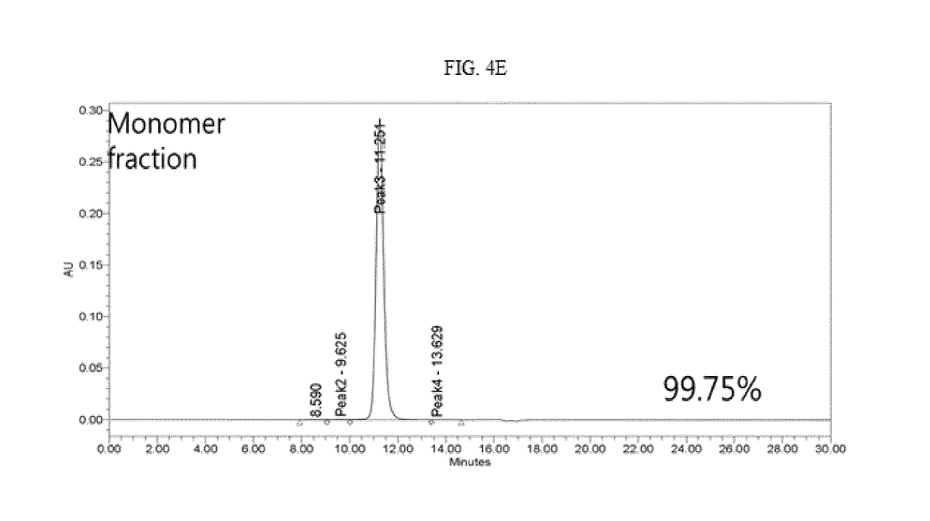

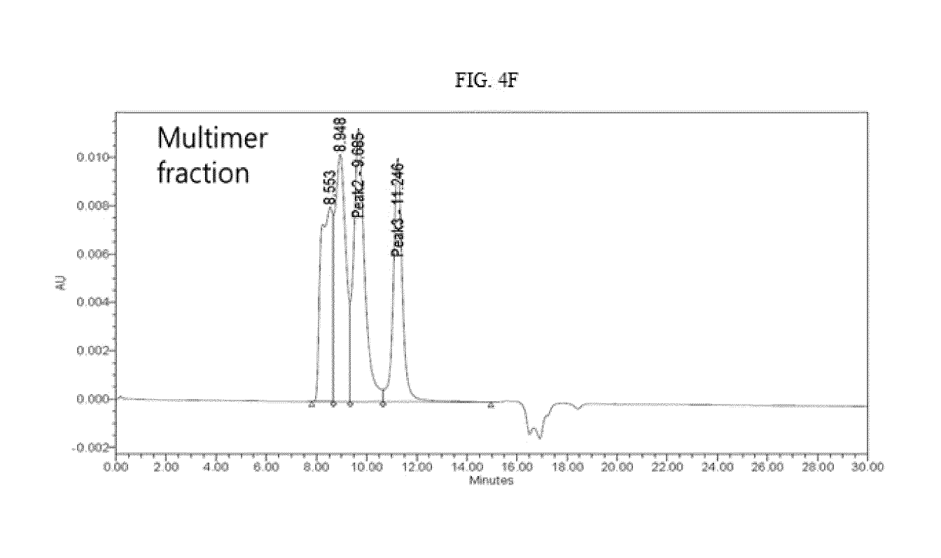

The results (fractions) of a SEC-HPLC chromatogram (using TSK G3000 swxl column; TOSHO Inc.) for the two conditions are shown in FIGS. 4A and 4B. As shown in FIGS. 4A and 4B, in the experiment using continuous pH gradient, the target protein (antibody) was eluted together with polymers; whereas in the experiment using discontinuous pH gradient, the target protein (antibody) was effectively separated from the polymers depending on pH change. In particular, when the pH of the wash buffer is from 5.4 to 5.6, completely no polymers bound to the resin, whereas all the target protein (antibody) completely bound to the resin without loss. In addition, when the pH of the elution buffer is 7.1, the highest yield was achieved.

2.4. Effect of Impurities Including Polymers on the Antibody Activity

The pure fractions (FIG. 4A: Fraction #1; FIG. 4B: Monomer Fraction) and the impurity-containing fractions (FIG. 4A: Fraction #2; FIG. 4B: Multimer Fraction), which were separated from Example 2.3, were provided. The Akt phosphorylation activity (agonism) and c-Met degradation activity (efficacy) were measured when a cell was treated with each of the fractions, to examine the effects of the impurities (e.g., dimers or multimers (more than dimer) of antibody) on the activity of the antibody.

The c-Met degradation activity (efficacy) was measured by the following method. Using the fact that the antibody binds to c-Met thereby inducing intracellular internalization and degradation of c-Met, the increase and decrease of the total amount of c-Met was measured to examine the efficacy of the antibody. Since it has been known that the binding between c-Met and HGF promotes the growth of cancer cells, it can be considered that the growth of cancer cells is lowered, when the total amount of c-Met is decreased. The total amount of c-Met was measured by quantitative ELISA method. The ELISA was performed using the human total HGF R/c-Met ELISA kit (R&D systems). As the cancer cell, gastric cancer cell line MKN45 (JCRB0254) was used. MKN45 cells (2×10⁵ cells/ml) and 5 μg/ml of the anti-c-Met antibody L3-1Y/IgG2 (Reference Example 1) were mixed and cultured (RPMI (Gibco), 37° C., 5% CO₂). 24 hours after, the ELISA was performed. Finally, the culture was reacted using Super Aquablue (eBiosciences), and the obtained colorimetric signals were measured at 450 nm as OD value. The value of the group treated with the anti-c-Met antibody L3-1Y/IgG2 was calculated compared to the value of the group treated with no anti-c-Met antibody L3-1Y/IgG2 (which is assumed as 100%).

The level of the Akt phosphorylation (agonism) was measured by quantitative ELISA method. The phosphorylation site of Akt is Ser 473. The phosphorylation at the site (Ser 473) was measured by ELISA using the PathScan phospho-AKT1 (Ser473) chemiluminescent Sandwich ELISA kit (Cell signaling). One day before the examination, 2×10⁵ cells/ml of renal cancer cell line Caki-1 (ATCC, HTB-46) was treated with a mixture of a serum-free medium (DMEM) and 5 ug/ml of the antibody for 30 minutes, and then subjected to the examination using the ELISA kit. The results were obtained using the instruments of Perkins Elmer Inc. For agonism comparison, another anti-c-Met antibody, 5D5 antibody (separated and purified from hybridoma of ATCC Cat. #HB-11895 obtained from American Type Culture Collection (ATCC, Manassas, Va.)) was used. When calculating the level of Akt phosphorylation, the level of Akt phosphorylation by 5D5 was considered as 100%, and the level of Akt phosphorylation induced by other anti-c-Met antibody was expressed by comparing to the level of 5D5. The cell functions controlled by Akt include cell proliferation, cell survival, cell size control, responsibility of available nutrients, intermediate metabolism, angiogenesis, tissue invasion, and the like, all of which stand for various features of cancer. Various oncoproteins and tumor suppressors cross-affect reciprocally on the Akt pathway, and carry out a sensitive control of the cell functions at a linking point of signal transduction and classical metabolic regulation. Therefore, as the level of phosphorylated Akt, which is an active form of Akt, becomes increased, the cancer cell is in the more active state. This is the reason to measure the inhibitory degree of Akt phosphorylation by the antibody.

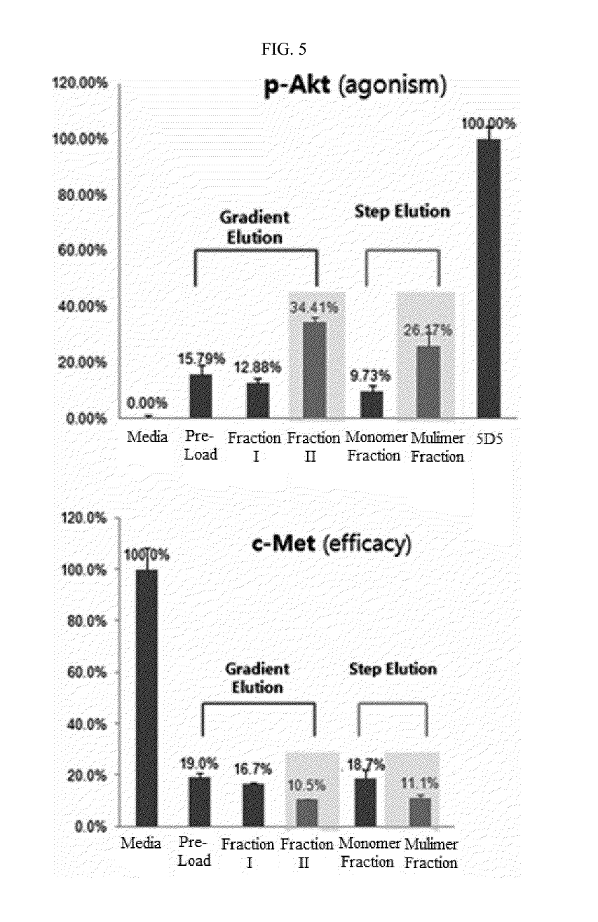

The obtained results were shown in FIG. 5. As shown in FIG. 5, the impurity fractions (fraction II or multimer fraction) show similar efficacy but considerably high agonism, compared to the pure fractions.

2.5. Conditions of Final CIEX

Based on the above experiment results, the final CIEX process was designed under the conditions as follows:

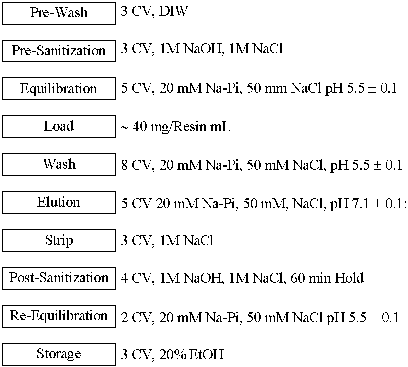

To continuously perform the CIEX process, it was designed so equilibration and loading is performed immediately after post-sanitization.

### Example 3

### Selection of Conditions of Affinity Chromatography

An examination for selecting proper conditions of affinity chromatography (AC) to efficiently separate the antibody from the culture solution, was performed.

As a design of Experiment (DoE), a response surface methodology (RSM) was employed. Optimal conditions of pH and salt concentration of wash and pH of elution buffer used in the AC process were selected.

The AC process consisted of a total of 12 steps from DIW wash step (pre-wash) to storage step, as illustrated in the following:

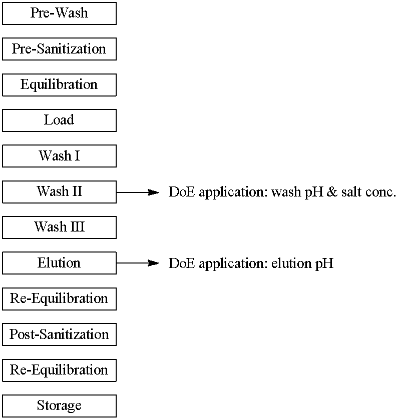

The experiment was carried out for the pH and salt concentration of wash II step, which was expected to affect the quality of the obtained eluate. At the same time, the pH condition of elution buffer was also screened.

The purification conditions for the performance of the experiment were summarized in Table 13:

TABLE 13

| LFR (cm/min) | VFR (ml/min) | Temp. (° C.) | Column |
|---|---|---|---|
| 12.7 | 10 | 20 to 22 | Tricorn 10/100 & |
| | | | 10 cm bed height |

The salt concentration and pH of the AC wash buffer II are factors having great effect on the quality of the obtained AC eluate, and the pH of the AC elution buffer is a factor having effect on the yield (recovery rate) of the protein and the quality of the recovered protein (formation of polymer, etc.). Therefore, the buffer conditions for the experiment were determined by combining the factors, and the determined conditions are summarized in Table 14:

TABLE 14

| Step | Buffer | Salt (mM NaCl) | pH | Vol.(CV) |
|---|---|---|---|---|
| AC Wash buffer I | 20 mM Sodium phosphate dibasic | — | 7.5 | 3 |
| AC Wash buffer II | 20 mM Sodium phosphate dibasic | 0 - 1000 | 4.5 ~ 7.5 | 3 |
| AC Elution I | 20 mM Citric acid | | 2.5 ~ 3.5 | 3 |

The obtained results are shown in Table 15:

TABLE 15

| Factor | Wash_pH | Wash_salt | Elution_pH | Wash HCP | Elution HCP | Purity | yield |
|---|---|---|---|---|---|---|---|
| Run No. | pH 4.5 ~ 7.5 | NaCl 0 ~ 1 M | pH 2.5 - 3.5 | (ng/ml) | (ng/ml) | (%) | (%) |
| 1 | 6.0 | 0.5 | 3.00 | 214.60 | 264.89 | 99.08 | 76.51 |
| 2 | 7.5 | 1.0 | 2.50 | 165.44 | 188.57 | 88.29 | 77.87 |
| 3 | 4.5 | 0.0 | 2.50 | 89.52 | 479.82 | 81.59 | 75.52 |
| 4 | 7.5 | 0.0 | 3.50 | 0.00 | 194.05 | 99.63 | 74.03 |
| 5 | 6.0 | 0.5 | 3.00 | 202.00 | 297.10 | 99.07 | 69.58 |
| 6 | 4.5 | 1.0 | 3.50 | 314.40 | 158.02 | 98.78 | 76.30 |
| 7 | 7.5 | 1.0 | 3.50 | 331.44 | 90.63 | 99.16 | 123.19 |
| 8 | 4.5 | 1.0 | 2.50 | 195.32 | 271.28 | 48.05 | 74.65 |
| 9 | 6.0 | 0.5 | 3.00 | 140.52 | 188.03 | 99.09 | 77.44 |
| 10 | 4.5 | 0.0 | 3.50 | 101.68 | 449.73 | 98.44 | 78.60 |
| 11 | 7.5 | 0.0 | 2.50 | 74.84 | 587.80 | 92.74 | 82.20 |

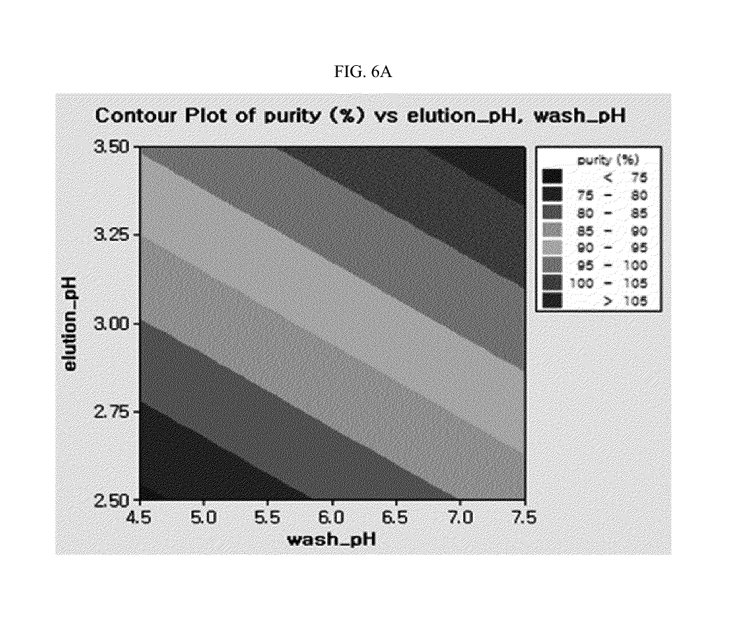

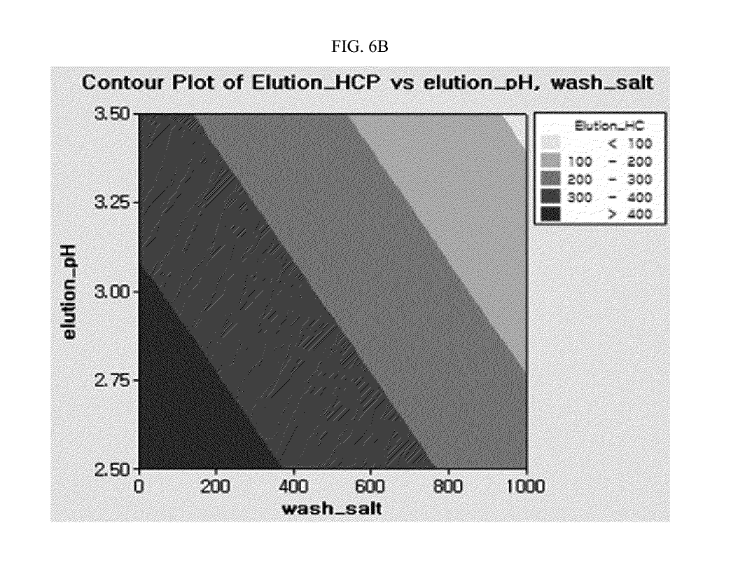

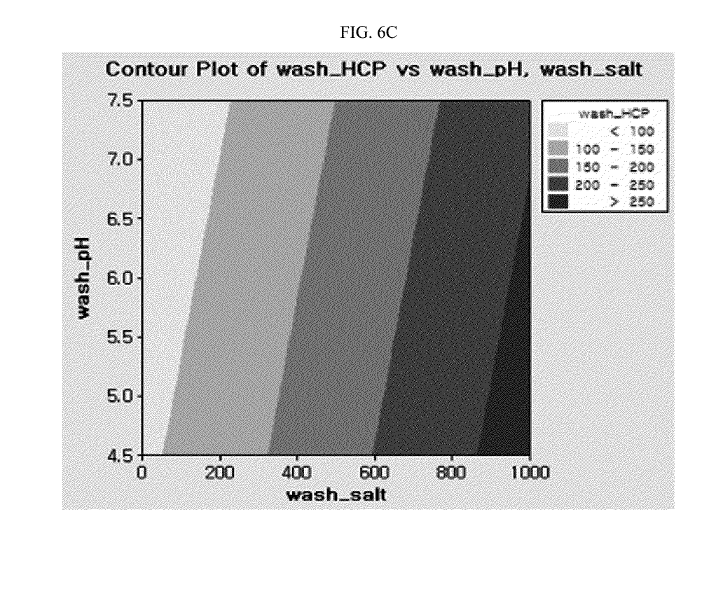

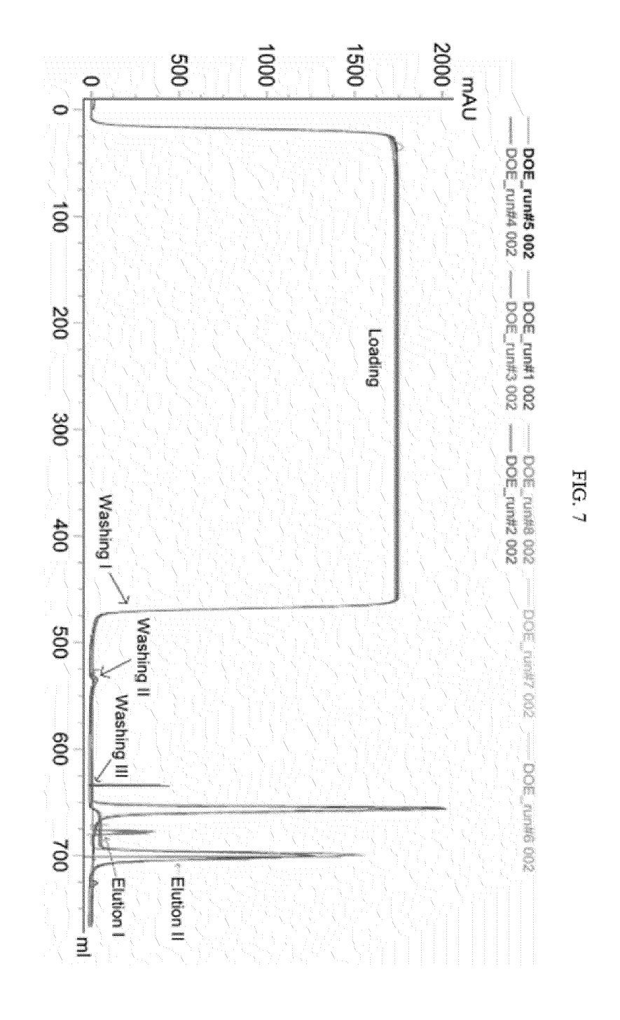

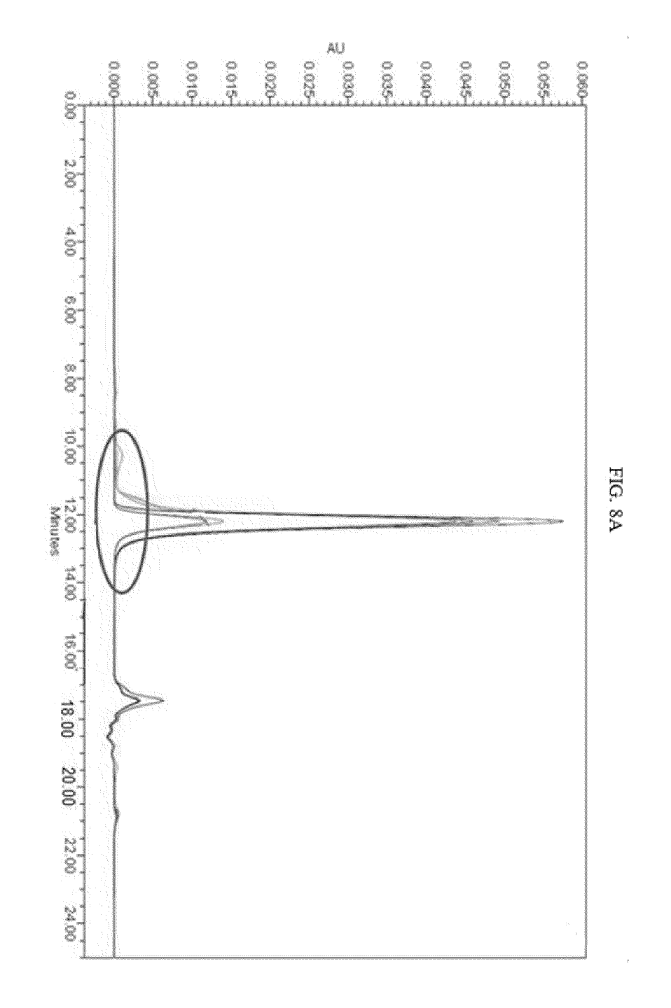

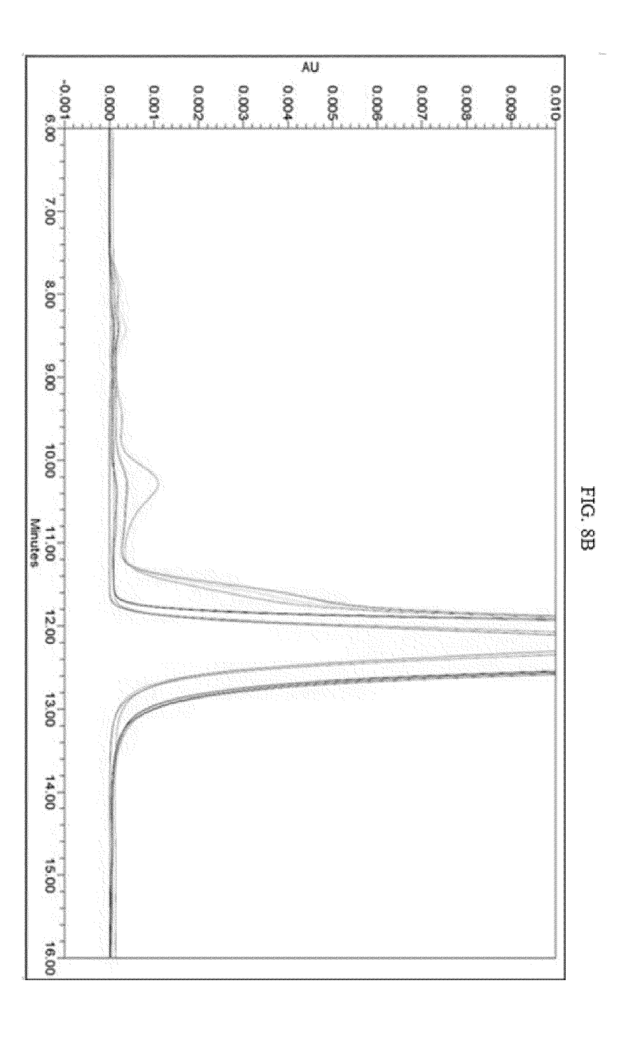

The results of Table 15 were analyzed and shown in FIG. 6, the results of the AC chromatogram were shown in FIG. 7, and the results of SEC-HPLC chromatogram (using TSK G3000 swxl column; TOSHO Inc.) which confirms the purity were shown in FIGS. 8A and 8B (magnified image of the circle in FIG. 8A). As shown in Table 15 and FIGS. 6 to 8B, as the pH and salt concentration of the wash buffer becomes increased, more excellent effect of washing can be achieved. The proper range of pH as an elution condition was pH 3.0 to 3.5. If the pH of the elution buffer is less than 3.0, dimers or multimers (more than dimer) was formed, and if the pH of the elution buffer is more than 3.5, the recovery rate of the protein is lowered.

Through the above results, the final AC process was determined as follows:

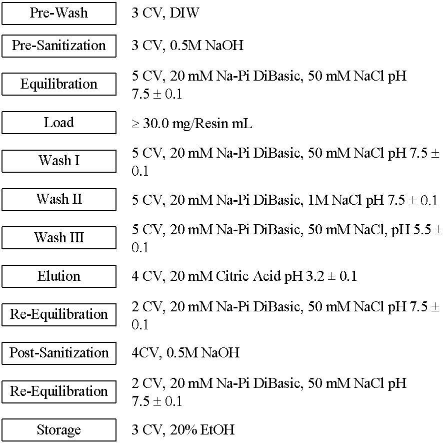

This process can be designed so as to be applied to a continuous column work for the production of 1000 L of clinical sample.

### Example 4

### Selection of Conditions of Anion-Exchange Chromatography

In addition to the conditions of the cation-exchange chromatography process selected in Example 2 and the conditions of the affinity chromatography process selected in Example 3, proper conditions of an anion-exchange chromatography (AIEX) process was further selected, to be used in the experiment showing that the efficacy of the antibody purification can be increased by removal host cell originated DNAs and host cell proteins (HCP).

The AIEX process was developed so that the antibody can be purified by binding to the resin impurities only (not the antibody protein). Considering the pI of the anti-c-Met antibody L3-1Y/IgG2 is 8.1 in calculation, three pH points (pH 6.5, 7.1, 7.5) were determined, and the quality of the sample depending on the pH of the loaded anti-c-Met antibody sample and the pH of the chase buffer was examined at the three points.

The AIEX process consisted of a total of 9 steps as follows, and the pH of the loaded antibody sample and the chase buffer was optimized at the above 3 points:

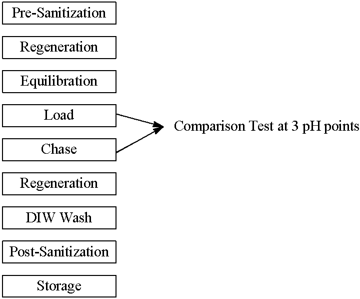

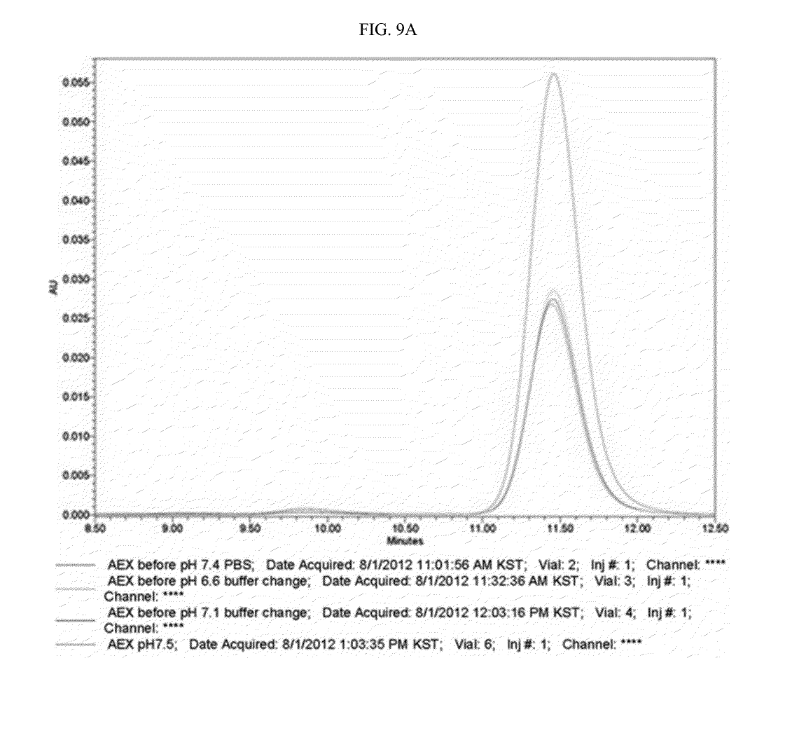

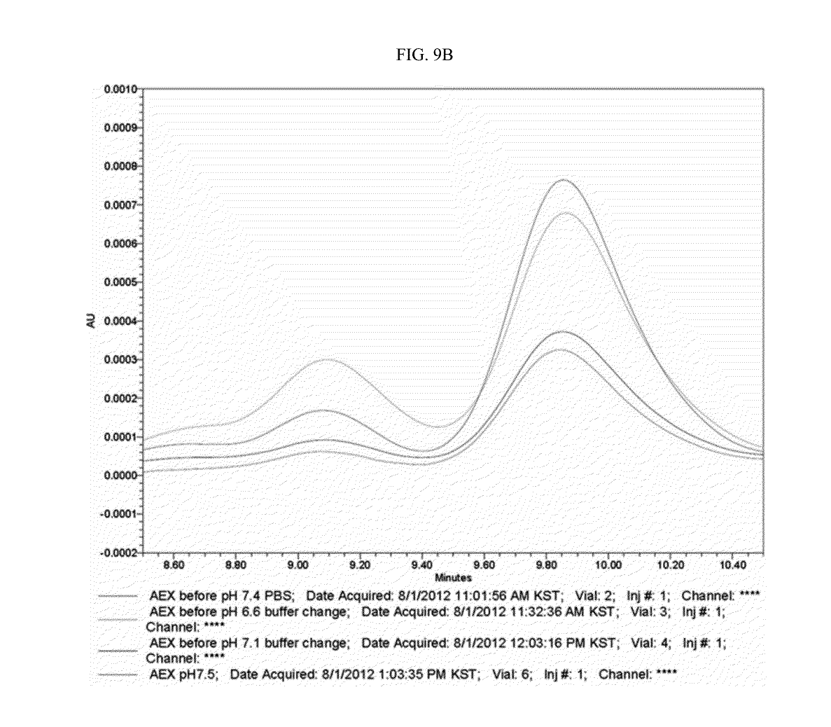

Assuming that the degree of formation of polymers may vary depending on the pH of the loaded sample, the pH of the antibody samples which is stored in PBS phase after the CIEX process was adjusted to pH 6.5, pH 7.1, and pH 7.5, respectively, using 1 M Sodium Phosphate dibasic or 1 M Sodium Phosphate monobasic. Thereafter, the samples were left at room temperature for 1 hour, and then the purity (degree of formation of polymers) of the samples was measured by SEC-HPLC (using TSK G3000 swxl column; TOSHO, Inc.). The obtained results are shown in FIGS. 9A and 9B (magnified image of the range of 8.6 to 10.4 minutes in FIG. 9A). As shown in FIGS. 9A and 9B, as the pH of the loaded sample becomes increased, the formation of polymers is effectively prevented. Therefore, considering the above results and connection with the preceding CIEX process, the pH of the loaded sample of the AIEX was determined as about 7.5.

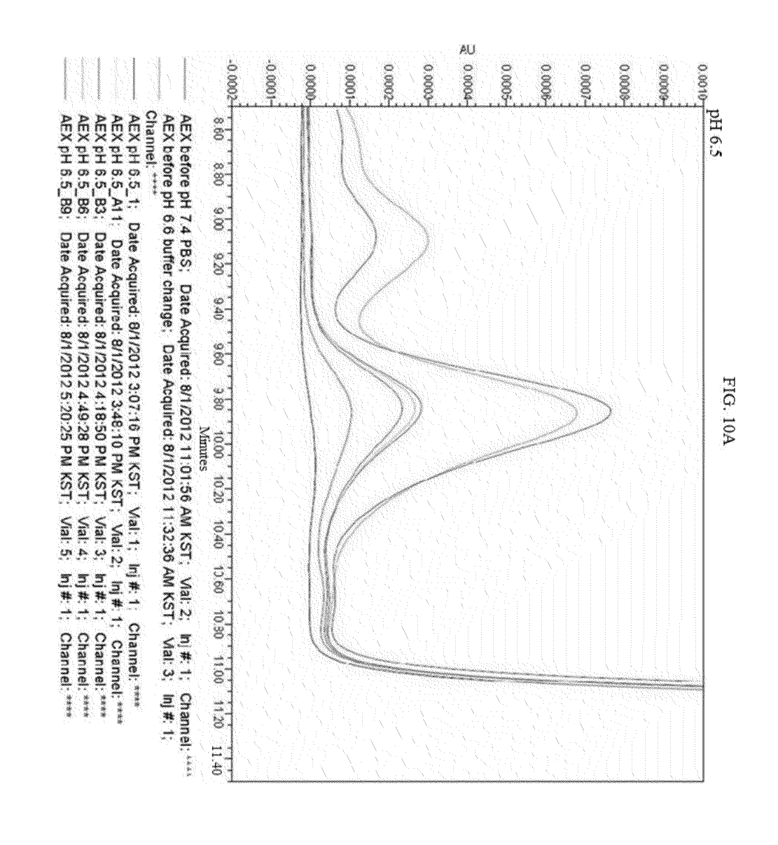

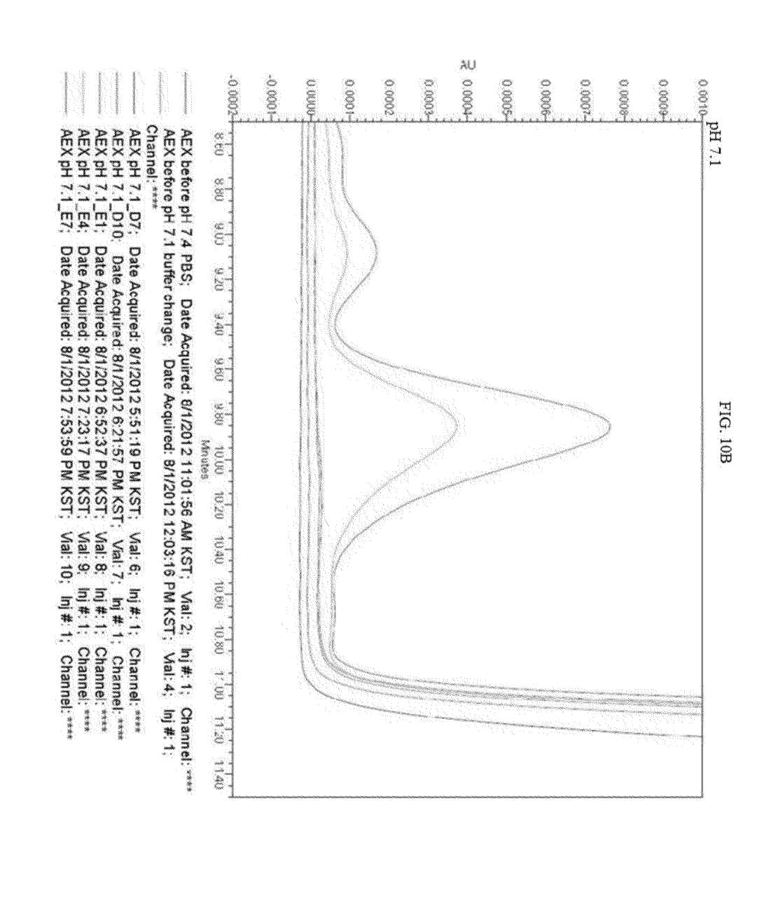

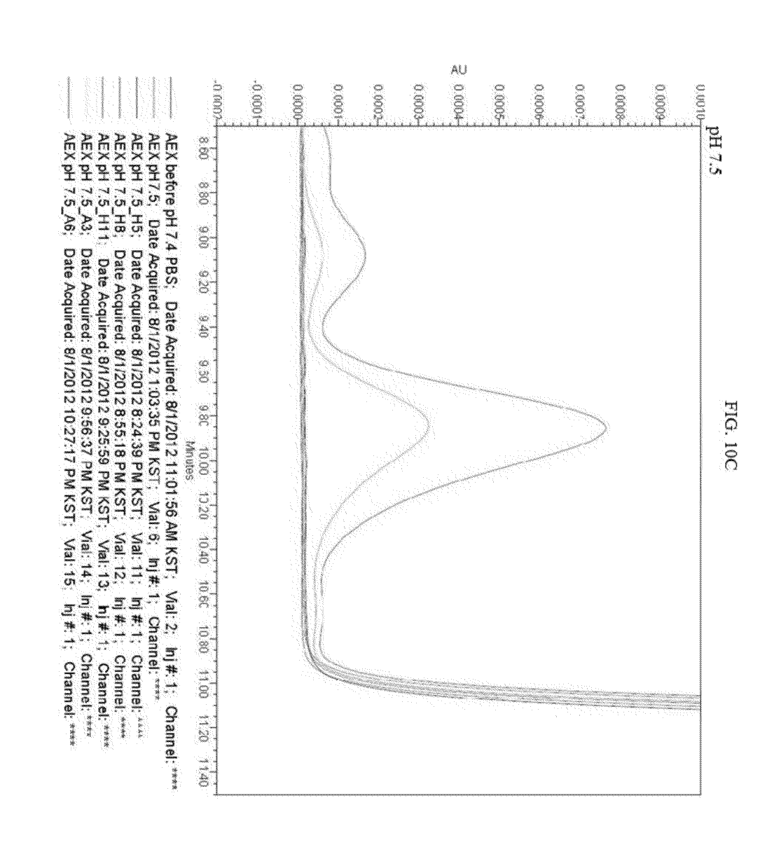

In addition, sodium phosphate monobasic and sodium phosphate dibasic were mixed and pH of the mixture was adjusted to pH 6.5, pH 7.1, or pH 7.5. Then each mixture was used as an AIEX chase buffer. After the sample was left at room temperature for 1 hour, and the purity (degree of formation of polymers) of the sample was measured by SEC-HPLC (using TSK G3000 swxl column; TOSHO Inc.). The obtained results are shown in FIGS. 10A to 10C. FIG. 10A shows the result at pH 6.5, FIG. 10B shows the result at pH 7.1, and FIG. 10C shows the result at pH 7.5. As shown in FIGS. 10A to 10C, as the pH of the chase buffer becomes higher, the purity of the antibody is increased and other impurities are also increased. Therefore, the proper pH of the chase buffer was determined as 6.5.

Based on the above experiment results, the final AIEX process was determined as follows:

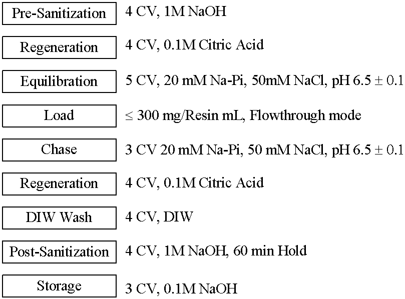

To continuously perform the AIEX process, it can be designed so that the equilibration and loading can be performed immediately after the post-sanitization.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of purifying an anti-c-Met antibody from an anti-c-Met antibody-containing sample, the method comprising performing an affinity chromatography step, a cation-exchange chromatography step, and an anion-exchange chromatography step on the anti-c-Met antibody-containing sample, wherein the cation-exchange chromatography step is performed under at least one condition selected from the group consisting of:
a condition that anti-c-Met antibody-containing sample loaded onto a cation-exchange chromatography material during the cation exchange chromatography step has a conductivity of about 5.5 mS/cm or less;
a condition that the cation-exchange chromatography step uses a wash buffer with a conductivity of about 7.0 mS/cm or less; and
a condition that the cation-exchange chromatography step use an elution buffer with a conductivity of about 7.6 mS/cm or more.

2. The method of claim 1, wherein the conductivity of the anti-c-Met antibody-containing sample loaded onto the cation-exchange chromatography material, the conductivity of the wash buffer, or the conductivity of the elution buffer is adjusted by adjusting salt concentration, pH, or a combination thereof, of the antibody sample, the wash buffer, or the elution buffer, respectively.

3. The method of claim 2, wherein the cation-exchange chromatography step is performed under at least one condition selected from the group consisting of:
a condition that the salt concentration of the anti-c-Met antibody-containing sample loaded to the cation-exchange chromatography is about 50 mM or less;
a condition that the pH of the anti-c-Met antibody-containing sample loaded onto the cation-exchange chromatography material during the cation exchange chromatography step is about 5.5 or less;
a condition that the salt concentration of the wash buffer used in the cation-exchange chromatography step is about 50 mM or less;
a condition that the pH of the wash buffer used in the cation-exchange chromatography step is about 5.2 to 5.8;
a condition that the salt concentration of the elution buffer used in the cation-exchange chromatography step is about 50 mM or less; and
a condition that the pH of the elution buffer used in the cation-exchange chromatography step is about 6.6 to 7.4.

4. The method of claim 2, wherein the salt concentration is adjusted by adding at least one selected from the group consisting of sodium chloride, magnesium sulfate, calcium chloride, ammonium sulfate, magnesium chloride, potassium chloride, and sodium sulfate, or any combination thereof to the antibody sample, the wash buffer, or the elution buffer.

5. The method of claim 2, wherein the wash buffer comprises at least one selected from the group consisting of phosphate compounds, acetate compounds, citrate compounds, carbonate compounds, HEPES (4-(2-hydroxyethyl)-1-piperazineethanesulfonic acid), MOPS (3-(N-morpholino)propanesulfonic acid), Tris, Bis-Tris, and MES (2-(N-morpholino)ethanesulfonic acid), wherein the pH of the wash buffer is about 5.2 to 5.8, and
the elution buffer comprises at least one selected from the group consisting of phosphate compounds, acetate compounds, citrate compounds, carbonate compounds, HEPES (4-(2-hydroxyethyl)-1-piperazineethanesulfonic acid), MOPS (3-(N-morpholino)propanesulfonic acid), Tris, Bis-Tris, and MES (2-(N-morpholino)ethanesulfonic acid), wherein the pH of the elution buffer is about 6.6 to 7.4.

6. The method of claim 1, wherein the affinity chromatography step is performed using an elution buffer with a pH of about 3.0 to 3.5.

7. The method of claim 1, wherein pH of the anti-c-Met antibody-containing sample loaded onto an anion-exchange chromatography material during the anion exchange chromatography step is about 6.5 to 9.

8. The method of claim 1, wherein the anion-exchange chromatography step is performed using a chase buffer with a pH of about 6 to 7.

9. The method of claim 1, wherein the method further comprises a virus inactivation step, a nanofiltration step, an ultrafiltration step, and a diafiltration step.

10. The method of claim 1, wherein the anti-c-Met antibody has an isoelectric point (pI) ranging from about 8 to about 8.5.

11. The method of claim 1, wherein the anti-c-Met antibody is an anti-c-Met antibody that recognizes or binds to 5 or more contiguous amino acid in SEMA domain (SEQ ID NO: 79) of c-Met.

12. The method of claim 1, wherein the anti-c-Met antibody comprises:
(i) at least one heavy chain complementarity determining region (CDR) selected from the group consisting of (a) a CDR-H1 comprising SEQ ID NO: 4; (b) a CDR-H2 comprising SEQ ID NO: 5, SEQ ID NO: 2, or 8-19 consecutive amino acids of SEQ ID NO: 2 including the 3^{rd} to 10^{th} positions of the amino acid sequence of SEQ ID NO: 2; and (c) a CDR-H3 comprising SEQ ID NO: 6, SEQ ID NO: 85, or 6-13 consecutive amino acids of SEQ ID NO: 85 including the 1^{st} to 6^{th} positions of the amino acid sequence of SEQ ID NO: 85, or a heavy chain variable region comprising the at least one heavy chain complementarity determining region;
(ii) at least one light chain complementarity determining region (CDR) selected from the group consisting of (a) a CDR-L1 comprising SEQ ID NO: 7, (b) a CDR-L2 comprising SEQ ID NO: 8, and (c) a CDR-L3 comprising SEQ ID NO: 9, SEQ ID NO: 15, SEQ ID NO: 86, or 9-17 consecutive amino acids of SEQ ID NO: 89 including the 1^{st} to 9^{th} positions of the amino acid sequence of SEQ ID NO: 89, or a light chain variable region including the at least one light chain complementarity determining region;
(iii) a combination of the at least one heavy chain complementarity determining region and at least one light chain complementarity determining region; or
(iv) a combination of the heavy chain variable region and the light chain variable region.

13. An anti-c-Met antibody agent prepared by the method of claim 1, wherein the purity of the anti-c-Met antibody agent is about 95% or more, the amount of polymers comprising at least two monomers in the anti-c-Met antibody agent is about 1%(w/w) or less, and the amount of host cell proteins in the anti-c-Met antibody agent is about 4 ppm or less.

14. An anti-c-Met antibody agent of claim 13, wherein the anti-c-Met antibody has an isoelectric point (pI) ranging from about 8 to about 8.5.

15. An anti-c-Met antibody agent of claim 13, wherein the anti-c-Met antibody is an anti-c-Met antibody that recognizes or binds to 5 or more contiguous amino acid in SEMA domain (SEQ IS NO: 79) of c-Met.

16. An anti-c-Met antibody agent of claim 15, wherein the anti-c-Met antibody comprises:
(i) at least one heavy chain complementarity determining region (CDR) selected from the group consisting of (a) a CDR-H1 comprising SEQ ID NO: 4; (b) a CDR-H2 comprising SEQ ID NO: 5, SEQ ID NO: 2, or 8-19 consecutive amino acids of SEQ ID NO: 2 including the 3^{rd} to 10^{th} positions of the amino acid sequence of SEQ ID NO: 2; and (c) a CDR-H3 comprising SEQ ID NO: 6, SEQ ID NO: 85, or 6-13 consecutive amino acids within of SEQ ID NO: including the 1^{st} to 6^{th} positions of the amino acid sequence of SEQ ID NO: 85, or a heavy chain variable region comprising the at least one heavy chain complementarity determining region;
(ii) at least one light chain complementarity determining region (CDR) selected from the group consisting of (a) a CDR-L1 comprising SEQ ID NO: 7, (b) a CDR-L2 comprising SEQ ID NO: 8, and (c) a CDR-L3 comprising SEQ ID NO: 9, SEQ ID NO: 15, SEQ ID NO: 86, or 9-17 consecutive amino acids of SEQ ID NO: 89 including the 1^{st} to 9^{th} positions of the amino acid sequence of SEQ ID NO: 89, or a light chain variable region including the at least one light chain complementarity determining region;
(iii) a combination of the at least one heavy chain complementarity determining region and at least one light chain complementarity determining region; or
(iv) a combination of the heavy chain variable region and the light chain variable region.

17. A method of purifying a protein from a protein-containing sample, the method comprising performing an affinity chromatography step, a cation-exchange chromatography step, and an anion-exchange chromatography step on the protein-containing sample,
wherein the protein has an isoelectric point (pI) ranging from about 8 to about 8.5, and
the cation-exchange chromatography step is performed under at least one condition selected from the group consisting of:
(1) a condition that the protein containing sample loaded onto a cation-exchange chromatography material during the cation exchange chromatography step has a conductivity of about 5.5 mS/cm or less;
(2) a condition that the cation-exchange chromatography step uses a wash buffer with a conductivity of about 7.0 mS/cm or less; and
(3) a condition that the cation-exchange chromatography step uses an elution buffer with a conductivity of about 7.6 mS/cm or more.
